SEQUENCE LISTING (62 sequences)

SEQ ID NO: 1 - RNA, 8 nt, Artificial
  Chemically Synthesized
agguragu

SEQ ID NO: 2 - RNA, 7 nt, Artificial
  Chemically Synthesized
ynyurac

SEQ ID NO: 3 - DNA, 258 bp, Artificial
  Chemically Synthesized
gtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttccacatctttatattttgcaccacattcaacactgtagcggccgc

SEQ ID NO: 4 - DNA, 270 bp, Artificial
  Chemically Synthesized
ccaactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccag

SEQ ID NO: 5 - DNA, 23 bp, Artificial
  Chemically Synthesized
taatacgactcactatagggaga

SEQ ID NO: 6 - DNA, 23 bp, Artificial
  Chemically Synthesized
atttaggtgacactatagaagng

SEQ ID NO: 7 - DNA, 23 bp, Artificial
  Chemically Synthesized
aattaaccctcactaaagggaga

SEQ ID NO: 8 - DNA, 81 bp, Artificial
  Chemically Synthesized
atgaagtgggtaacctttctcctcctcctcttcgtctccggctctgctttttccaggggtgtgtttcgccgagaagcaccc

SEQ ID NO: 9 - DNA, 82 bp, Artificial
  Chemically Synthesized
gggtgcttctcggcgaaacacacccctggaaaaagcagagccggagacgaagaggaggaggagaaaggttacccacttcatg

SEQ ID NO: 10 - DNA, 81 bp, Artificial
  Chemically Synthesized
atgaagtgggtaacctttatttcccttctttttctctttagctcggcttattccaggggtgtgtttcgtcgagatgcaccc

SEQ ID NO: 11 - DNA, 82 bp, Artificial
  Chemically Synthesized
gggtgcatctcgacgaaacacacccctggaataagccgagctaaagagaaaaagaagggaaataaaggttacccacttcatg

SEQ ID NO: 12 - DNA, 24 bp, Artificial
  Chemically Synthesized
ccccagagcccctgggatcgagtg

SEQ ID NO: 13 - DNA, 42 bp, Artificial
  Chemically Synthesized
ctagaagcttcccactttggaaacgtttattctgagcaccgg

SEQ ID NO: 14 - DNA, 32 bp, Artificial
  Chemically Synthesized
ctagggatccgttttatgttttttcatctctg

SEQ ID NO: 15 - DNA, 35 bp, Artificial
  Chemically Synthesized
ctaggcggccgcaggcctttgaaatgttgttctcc

SEQ ID NO: 16 - DNA, 34 bp, Artificial
  Chemically Synthesized
ctaggctagcacctttcctatcaaccccactagc

SEQ ID NO: 17 - DNA, 35 bp, Artificial; identical to SEQ ID NO: 15
  Chemically Synthesized

SEQ ID NO: 18 - DNA, 897 bp, Artificial
  Chemically Synthesized
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 19 - DNA, 38 bp, Artificial
  Chemically Synthesized
ctaggctagcagagactgcgagaaggaggtcccccacg

SEQ ID NO: 20 - DNA, 42 bp, Artificial; identical to SEQ ID NO: 13
  Chemically Synthesized

SEQ ID NO: 21 - DNA, 6 bp, Artificial
  Chemically Synthesized
gctagc

SEQ ID NO: 22 - DNA, 6 bp, Artificial
  Chemically Synthesized
aagctt

SEQ ID NO: 23 - DNA, 15 bp, Artificial
  Chemically Synthesized
gagatgcgcgaccgc

SEQ ID NO: 24 - DNA, 59 bp, Artificial
  Chemically Synthesized
ctagcacgagctgcaagagaagctgagcccactgggcgaggagatgtgcgaccgcgcgc

SEQ ID NO: 25 - DNA, 15 bp, Artificial
  Chemically Synthesized
gagatgtgcgaccgc

SEQ ID NO: 26 - DNA, 6 bp, Artificial
  Chemically Synthesized
gctagc

SEQ ID NO: 27 - DNA, 53 bp, Artificial
  Chemically Synthesized
ccgcggtactaactcaatttttttttttttttttttaattaacagcccctaaa

SEQ ID NO: 28 - DNA, 9 bp, Artificial
  Chemically Synthesized
ccggtgagt

SEQ ID NO: 29 - DNA, 6 bp, Artificial
  Chemically Synthesized
cagaag

SEQ ID NO: 30 - DNA, 9 bp, Artificial
  Chemically Synthesized
caagtaagg

SEQ ID NO: 31 - DNA, 6 bp, Artificial
  Chemically Synthesized
tagaag

SEQ ID NO: 32 - DNA, 9 bp, Artificial
  Chemically Synthesized
aaggtatgg

SEQ ID NO: 33 - DNA, 6 bp, Artificial
  Chemically Synthesized
gctagc

SEQ ID NO: 34 - DNA, 6 bp, Artificial
  Chemically Synthesized
ccgcgg

SEQ ID NO: 35 - DNA, 47 bp, Artificial
  Chemically Synthesized
tactaactcaatttttttttttttttttttaattaacagcccctaaa

SEQ ID NO: 36 - DNA, 54 bp, Artificial
  Chemically Synthesized
atgaaagctgcggtgctgaccttggccgtgctcttcctgacggggagccaggct

SEQ ID NO: 37 - DNA, 18 bp, Artificial
  Chemically Synthesized
cggcatttctggcagcaa

SEQ ID NO: 38 - DNA, 66 bp, Artificial
  Chemically Synthesized
gatgaacccccccagagcccctgggatcgagtgaaggacctggccactgtgtacgtggatgtgctc

SEQ ID NO: 39 - DNA, 22 bp, Artificial
  Chemically Synthesized
ccgagaagcacccccccagagc

SEQ ID NO: 40 - DNA, 859 bp, Artificial
  Chemically Synthesized
atgaagtgggtaacctttatttcccttctttttctctttagctcggcttattccaggggtgtgtttcgtcgagatgcacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 41 - DNA, 66 bp, Artificial
  Chemically Synthesized
ggaaacagacaagagtgaggttgctcatcggtttaaagatttgggagaagaaaatttcaaagcctt

SEQ ID NO: 42 - DNA, 209 bp, Artificial
  Chemically Synthesized
atggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacag

SEQ ID NO: 43 - DNA, 700 bp, Artificial
  Chemically Synthesized
gtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcc

SEQ ID NO: 44 - DNA, 67 bp, Artificial
  Chemically Synthesized
cttgcccagacaagagtgaggttgctcatcggtttaaagatttgggagaagaaaatttcaaagcctt

SEQ ID NO: 45 - DNA, 79 bp, Artificial
  Chemically Synthesized
atgaagtgggtaacctttctcctcctcctcttcgtctccggctctgctttttccaggggtgtgtttcgccgagaagcac

SEQ ID NO: 46 - DNA, 123 bp, Artificial
  Chemically Synthesized
gtttttagtatagcatggtcgagcaggcaggccctatgagaccgtaataattcaactgtatccaacgtaatttgagtcattctgcctagcatttttttttaattaaaagaaatttaaagaaac

SEQ ID NO: 47 - DNA, 278 bp, Artificial
  Chemically Synthesized
cgtttccacaacatatttaaagattgatgaagacaactaactgtaatatgctgctttttgttcttctcttcactgacctaagctactccctgaagatgccagttcccgatcgttacaggaaaatctgaaaaagcttgcaatggttcctctctgctgcactcaaagttatattttttcaccaacattattatttttaaaacccgttaagtgtttatatctgtgcattcaaactcaagatttagtgtttctgtcatgtttgaaaatatctactaagaaaa

SEQ ID NO: 48 - DNA, 150 bp, Artificial
  Chemically Synthesized
gtttttagtatagcatggtcgagcaggcaggccctatgagaccgtaataaattcaactgtatccaacgtaatttgagtcattctgcctagcatttttttttaattaaaagaaatttaaagctaagctttcaaaatcccccattattaaac

SEQ ID NO: 49 - DNA, 149 bp, Artificial
  Chemically Synthesized
gtttggttcctctctgctacactcaaagttatattctttcaccaacattattatttttaaaacccgttaagtgtttatatctgtgcattcaaactcaagatttagtgtttctgtcatgtttgtaaatatctactaagacaatggtaaac

SEQ ID NO: 50 - DNA, 140 bp, Artificial
  Chemically Synthesized
tttttagtatagcatggtcgagcaggcaggccctatgagaccgtaataaattcaactgtatccaacgtaatttgagtcattctgcctagcatttttttttaattaaaagaaatttaaagctaagctttcaaaatccccca

SEQ ID NO: 51 - DNA, 161 bp, Artificial
  Chemically Synthesized
tggaaactccatttataaaactagtaacacacacttttaatttttagtatagcatggtcgagcaggcaggccctatgagaccgtaataaattcaactgtatccaacgtaatttgagtcattctgcctagcatttttttttaattaaaagaaatttaaagct

SEQ ID NO: 52 - DNA, 343 bp, Artificial
  Chemically Synthesized
tgaaatgttgttctcccaaatcattataccgatgggcgatctcactcttgtctgtggaaacagggagagaaaaaccacacacatatttaaagattgatgaagacaactaactgtaatatgctgctttttgttcttcccttcactgacctaagctactccctgaagatgccagttcccgatcgttacaggaaaatctgaaggtggcaatggttcctctctgctacactcaaagttatattttttcaccaacattattatttttaaaacccgttaagtgtttatatctgtgcattcagactcaagatttagtgtttctgtcatgtttgtaaatatctactaagac

SEQ ID NO: 53 - DNA, 206 bp, Artificial
  Chemically Synthesized
tagcatggtcgagcaggcaggccctatgagaccgtaatagattcaactgtatccaacgtaaatttgagtcattctgcctagcatttttttttaattaaaagaaatttaaagctaagctttcaaaatcccccattattgtcatcaaagataccaaaaatatatcaataatataaccacctaagggttctcagatgcaaataataaca

SEQ ID NO: 54 - DNA, 114 bp, Artificial
  Chemically Synthesized
gtaacacacacttttaatttttagtatagcatggtcgagcaaggcaggccctatgagaccgtaataaattcaactgtatccaacgtaatttgagtcattctgcctagcattttt

SEQ ID NO: 55 - DNA, 205 bp, Artificial
  Chemically Synthesized
agcatggtcgagcaggcaggccctatgagaccgtaatagattcaactgtatccaacgtaatttgagtcattctgcctagcatttttttttaattaaaagaaatttaaagctaagctttcaaaatcccccattattgtcatcaaagataccaaaaatatatcaataatataaccacctaagggttctcagatgcaaataataacaa

SEQ ID NO: 56 - DNA, 6 bp, Artificial
  Chemically Synthesized
gctagc

SEQ ID NO: 57 - DNA, 44 bp, Artificial
  Chemically Synthesized
ccgcggacgatctcatattctatcgtcgaatactaactcaattt

SEQ ID NO: 58 - DNA, 39 bp, Artificial
  Chemically Synthesized
ttttttttttttttttaattaacagccccccagagcccc

SEQ ID NO: 59 - DNA, 52 bp, Artificial
  Chemically Synthesized
gggtgtgtttcgccgagaagcacccccccagagcccctgggatcgagtgaag

SEQ ID NO: 60 - DNA, 54 bp, Artificial
  Chemically Synthesized
aggggtgtgtttcgccgagaagcacccccccagagcccctgggatcgagtgaag

SEQ ID NO: 61 - DNA, 64 bp, Artificial
  Chemically Synthesized
nnnnnnnttttttccaggggtgtgtttcgccgagaagcacccccccagagcccctgggatcgag

SEQ ID NO: 62 - DNA, 79 bp, Artificial
  Chemically Synthesized
atgaagtgggtaacctttatttcccttctttttctctttagctcggcttattccaggggtgtgtttcgtcgagatgcac

(19) United States
(12) Patent Application Publication
(10) Pub. No.: US 2006/0177933 A1
(43) Pub. Date: Aug. 10, 2006

(54) Expression of apoA-1 and variants thereof using spliceosome mediated RNA trans-splicing

(76) Inventors: Madaiah Puttaraju, Germantown, MD (US); Edward Otto, Reston, VA (US); Mariano A. Garcia-Blanco, Durham, NC (US); Gerard J. McGarrity, Gaithersburgh, MD (US); Gary F. Temple, Washington Grove, MD (US)

Correspondence Address: ARENT FOX PLLC, 1675 BROADWAY, NEW YORK, NY, 10019, US

(21) Appl. No.: 11/041,155

(22) Filed: Jan. 21, 2005

Related U.S. Application Data
(60) Provisional application No. 60/538,796, filed on Jan. 23, 2004.
(60) Provisional application No. 60/584,280, filed on Jun. 30, 2004.

(51) Int. Cl. C12N 15/867; C12N 5/08

(52) U.S. Cl. 435/456; 435/366; 435/370

(57) ABSTRACT

The present invention provides methods and compositions for generating novel nucleic acid molecules through targeted spliceosome mediated RNA trans-splicing that result in expression of an apoA-1 variant, the preferred embodiment referred to herein as the apoA-1 Milano variant. The compositions of the invention include pre-trans-splicing molecules (PTMs) designed to interact with a target precursor messenger RNA molecule (target pre-mRNA) and mediate a trans-splicing reaction resulting in the generation of a novel chimeric RNA molecule (chimeric RNA) capable of encoding the apoA-1 Milano variant. The expression of this variant protein results in protection against vascular disorders resulting from plaque build up, i.e., strokes and heart attacks. In particular, the PTMs of the present invention include those genetically engineered to interact with the apoA-1 target pre-mRNA so as to result in expression of the apoA-1 Milano variant. In addition, the PTMs of the invention include those genetically engineered to interact with the apoB or albumin or other specific target pre-mRNAs so as to result in expression of an apoB/apoA-1 and/or alb/apoA-1 wild type or Milano fusion protein thereby reducing apoB expression and simultaneously produce ApoA-1 function.

### CROSS-REFERENCE TO RELATED APPLICATIONS

The present application claims priority to U.S. Provisional Application Nos. 60/538,796, filed Jan. 23, 2004, and 60/584,280, filed Jun. 30, 2004, the disclosures of which are incorporated by reference in their entireties.

### INTRODUCTION

The present invention provides methods and compositions for generating novel nucleic acid molecules through targeted spliceosome mediated RNA trans-splicing that result in expression of wild type apoA-1 or variants such as, for example, the apoA-1 Milano variant. The compositions of the invention include pre-trans-splicing molecules (PTMs) designed to interact with a target precursor messenger RNA molecule (target pre-mRNA) and mediate a trans-splicing reaction resulting in the generation of a novel chimeric RNA molecule (chimeric RNA) capable of encoding the wild type apoA-1 or, variants, such as the Milano variant. The expression of this protein results in protection against cardiovascular disorders resulting from plaque build up, i.e., strokes and heart attacks.

In particular, the PTMs of the present invention include those genetically engineered to interact with the apoA-1 target pre-mRNA so as to result in expression of the apoA-1 Milano variant. In addition, the PTMs of the invention include those genetically engineered to interact with the apoB target pre-mRNA and/or any other selected target pre-mRNAs, so as to result in expression of an apoB/apoA-1 Milano fusion protein thereby reducing apoB expression and producing ApoA-1 Milano function. In addition, the present invention includes the use of other methods, such as trans-splicing ribozymes to create apoA-1 Milano chimeric mRNA and proteins. The compositions of the invention further include recombinant vector systems capable of expressing the PTMs of the invention and cells expressing said PTMs.

The methods of the invention encompass contacting the PTMs of the invention with an apoA-1 target pre-mRNA, and/or an apoB target pre-mRNA under conditions in which a portion of the PTM is trans-spliced to a portion of the target pre-mRNA to form a mRNA molecule wherein (i) expression of apoA-1 is substituted with expression of the apoA-1 Milano variant; and/or (ii) expression of apoB is substituted with expression of an apoB/apoA-1 Milano fusion protein and the level of apoB expression is simultaneously reduced. The methods of the invention also encompass contacting the PTMs of the invention with other target pre-mRNAs, which are highly expressed and encode efficiently secreted liver proteins, under conditions in which a portion of the PTM is trans-spliced to a portion of the target pre-mRNA to form a mRNA molecule wherein expression of the highly expressed protein is substituted with expression of the wild type apoA-1 or Milano variant. The compositions of the present invention may be administered in combination with other cholesterol lowering agents or lipid regulating agents. The methods and compositions of the present invention can be used to prevent or reduce the level of vascular plaque buildup that is normally associated with cardiovascular disease.

The albumin gene is highly expressed in the liver, thereby providing an abundant target pre-mRNA for targeting. The PTMs of the present invention include those genetically engineered to interact with an albumin target pre-mRNA so as to result in expression of wild type apoA-1, or apoA-1 variants such as the Milano variant. The methods of the invention encompass contacting such PTMs with an albumin target pre-mRNA under conditions in which a portion of the PTM is trans-spliced to a portion of the albumin target pre-mRNA to form a chimeric mRNA molecule wherein expression of albumin is substituted with expression of wild type apoA-1 or apoA-1 variants such the apoA-1 Milano variant.

### BACKGROUND OF THE INVENTION

### RNA Splicing

DNA sequences in the chromosome are transcribed into pre-mRNAs which contain coding regions (exons) and generally also contain intervening non-coding regions (introns). Introns are removed from pre-mRNAs in a precise process called cis-splicing (Chow et al., 1977, Cell 12:1-8; and Berget, S. M. et al., 1977, Proc. Natl. Acad. Sci. USA 74:3171-3175). Splicing takes place as a coordinated interaction of several small nuclear ribonucleoprotein particles (snRNP's) and many protein factors that assemble to form an enzymatic complex known as the spliceosome (Moore et al., 1993, in The RNA World, R. F. Gestland and J. F. Atkins eds. (Cold Spring Harbor Laboratory Press, Cold Spring Harbor, N.Y.); Kramer, 1996, Annu. Rev. Biochem., 65:367-404; Staley and Guthrie, 1998, Cell 92:315-326).

In most cases, the splicing reaction occurs within the same pre-mRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Natl. Acad. Sci. USA, 87:8879; Davis et al., 1995, J. Biol. Chem. 270:21813) and in plant mitochondria (Malek et al., 1997, Proc. Natl. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of splice leader trans-splicing, which is nearly identical to that of conventional cis-splicing, proceeds via two phosphoryl transfer reactions. The first causes the formation of a 2′-5′ phosphodiester bond producing a ‘Y’ shaped branched intermediate, equivalent to the lariat intermediate in cis-splicing. The second reaction, exon ligation, proceeds as in conventional cis-splicing. In addition, sequences at the 3′ splice site and some of the snRNPs which catalyze the trans-splicing reaction, closely resemble their counterparts involved in cis-splicing.

Trans-splicing refers to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Natl. Acad. Sci. USA 86:8020). In addition, trans-splicing of c-myb pre-mRNA has been demonstrated (Vellard, M. et al. Proc. Natl. Acad. Sci., 1992 89:2511-2515) and RNA transcripts from cloned SV40 trans-spliced to each other were detected in cultured cells and nuclear extracts (Eul et al., 1995, EMBO. J 14:3226). However, naturally occurring trans-splicing of mammalian pre-mRNAs is thought to be a rare event (Flouriot G. et al., 2002 J. Biol. Chem: Finta, C. et al., 2002 J Biol Chem 277:5882-5890).

In vitro trans-splicing has been used as a model system to examine the mechanism of splicing by several groups (Konarska & Sharp, 1985, Cell 46:165-171 Solnick, 1985, Cell 42:157; Chiara & Reed, 1995, Nature 375:510; Pasman and Garcia-Blanco, 1996, Nucleic Acids Res. 24:1638). Reasonably efficient trans-splicing (30% of cis-spliced analog) was achieved between RNAs capable of base pairing to each other, splicing of RNAs not tethered by base pairing was further diminished by a factor of 10. Other in vitro trans-splicing reactions not requiring obvious RNA-RNA interactions among the substrates were observed by Chiara & Reed (1995, Nature 375:510), Bruzik J. P. & Maniatis, T. (1992, Nature 360:692) and Bruzik J. P. and Maniatis, T., (1995, Proc. Natl. Acad. Sci. USA 92:7056-7059). These reactions occur at relatively low frequencies and require specialized elements, such as a downstream 5′ splice site or exonic splicing enhancers.

In addition to splicing mechanisms involving the binding of multiple proteins to the precursor mRNA which then act to correctly cut and join RNA, a third mechanism involves cutting and joining of the RNA by the intron itself, by what are termed catalytic RNA molecules or ribozymes. The cleavage activity of ribozymes has been targeted to specific RNAs by engineering a discrete “hybridization” region into the ribozyme. Upon hybridization to the target RNA, the catalytic region of the ribozyme cleaves the target. It has been suggested that such ribozyme activity would be useful for the inactivation or cleavage of target RNA in vivo, such as for the treatment of human diseases characterized by production of foreign of aberrant RNA. In such instances small RNA molecules are designed to hybridize to the target RNA and by binding to the target RNA prevent translation of the target RNA or cause destruction of the RNA through activation of nucleases. The use of antisense RNA has also been proposed as an alternative mechanism for targeting and destruction of specific RNAs.

Using the Tetrahymena group I ribozyme, targeted trans-splicing was demonstrated in E. coli. (Sullenger B. A. and Cech. T. R., 1994, Nature 341:619-622), in mouse fibroblasts (Jones, J. T. et al., 1996, Nature Medicine 2:643-648), human fibroblasts (Phylacton, L. A. et al. Nature Genetics 18:378-381) and human erythroid precursors (Lan et al., 1998, Science 280:1593-1596). For a review of clinically relevant technologies to modify RNA see Sullenger and Gilboa, 2002 Nature 418:252-8. The present invention relates to the use of targeted trans-splicing mediated by native mammalian splicing machinery, i.e., spliceosomes, to reprogram or alter the coding sequence of a targeted mRNA.

U.S. Pat. Nos. 6,083,702, 6,013,487 and 6,280,978 describe the use of PTMs to mediate a trans-splicing reaction by contacting a target precursor mRNA to generate novel chimeric mRNAs.

### Cardiovascular Disease

Cardiovascular disease (CVD) is the most common cause of death in the Western societies, and its prevalence is increasing worldwide. One of the strongest predictors of risk is the plasma concentration of high-density lipoprotein (HDL) or apolipoprotein A1 (apoA-1), the major protein component of HDL, which exhibits an inverse relationship with the development of atherosclerosis and coronary heart disease (Sirtori C R et al., 1999, Atherosclerosis 142:29-40; Genest J 2003, J Inherit. Metab. Dis. 26:267-287). ApoA-1 is the major apolipoprotein of HDL and is a relatively abundant plasma protein with a concentration of 1.0-1.5 mg/ml. ApoA-1 plays an important role in promoting the efflux of excess cholesterol from peripheral cells and tissues for transfer to the liver for excretion, a process called reverse cholesterol transport (RCT). Numerous in vitro and in vivo studies have demonstrated the protective effects of apoA-1 and HDL against atherosclerosis plaque development (Rubin E M, et al., Nature. 1991, 353:265-7; Plump A S et al., 1994 Proc Natl Acad. Sci. USA 91:9607-11; Paszty C, et al., 1994 J Clin Invest. 94:899-903; Duverger N et al., 1996, Circulation 94:713-7).

ApoA-1 Milano is one of a number of naturally occurring variants of wild type apoA-1. It was first identified in 1980 in an Italian family (Franceschini G et al., 1980, J. Clin. Invest. 66:892-900; Weisgraber K H et al., 1980 J Clin Invest. 66:901-907). To date 40 carriers have been identified and all are heterozygous. These carriers have low plasma HDL-cholesterol levels and moderately elevated levels of triglycerides, a condition that is usually associated with high-risk predictors for coronary heart disease. Despite severe reductions in plasma HDL-cholesterol levels and apoA-1 concentrations, the affected carriers do not develop coronary artery disease. In fact, infusions of the purified recombinant apoA-1 Milano or expression of apoA-1 Milano in rabbits and apoE deficient mice show protection against plaque formation and atherosclerosis (Ameli S et al., 1994, Circulation 90:1935-41; Soma M R et al., 1995 Cir. Res. 76:405-11; Shah P K et al., 1998 Circulation 97:780-5; Franceschini G et al., 1999, Arterioscler Thromb Vasc Biol. 19:1257-1262; Chiesa G et al., 2002, Cir. Res. 90:974-80; Chiesa G and Sirtori C, 2003, Curr. Opin. Lipdol. 14:159-163). Results from clinical trials, however have shown more modest levels of reduction. The degree of plaque reduction may be related to the limited number of doses and amounts of protein administered, and/or its duration in the circulation (pharmacokinetics).

Plasma apoA-1 is a single polypeptide chain of 243 amino acids, whose primary sequence is known (Brewer et al, 1978, Biochem. Biophys. Res. Commun. 80:623-630). ApoA-1 is synthesized as a 267 amino acid precursor in the cell. This preproapolipoproteinA-1 is first intracellularly processed by N-terminal cleavage of 18 amino acids to yield proapolipoproteinA-1, and then further cleavage of 6 amino acids in the plasma or the lymph by the activity of specific proteases to yield mature apolipoproteinA-1. The major structural requirement of the apoA-1 molecule is believed to be the presence of repeat units of 11 or 22 amino acids, presumed to exist in amphipathic helical conformation (Segrest et al., 1974, FEBS Lett 38:247-253). This structure allows for the main biological activities of apoA-1, i.e. lipid binding and lecithin:cholesterol acyltransferase (LCAT) activation.

Human apolipoproteinA1 Milano (apoA-1 Milano) is a natural variant of ApoA-1 (Weisgraber et al, 1980, J. Clin. Invest 66:901-907). In apoA-1 Milano the amino acid Arg173 is replaced by the amino acid Cys173. Since apoA-1 Milano contains one Cys residue per polypeptide chain, it may exist in a monomeric, homodimeric, or heterodimeric form. These forms are chemically interchangeable, and the term apoA-1 Milano does not, in the present context, discriminate between these forms. On the DNA level the variant form results from a C to T substitution in the gene sequence, i.e. the codon CGC changed to TGC, allowing the translation of a Cys instead of Arg at amino acid position 173. However, this variant of apoA-1 is one of the most interesting variants, in that apoA-1 Milano subjects are characterized by a remarkable reduction in HDL-cholesterol level, but without an apparent increased risk of arterial disease (Franceschini et al. 1980, J. Clin. Invest 66:892-900).

Another useful variant of apoA-1 is the Paris variant, where the arginine 151 is replaced with a cysteine.

The systemic infusion of ApoA-1 alone (Miyazaki et al. 1995, Arterioscler Thromb Vasc Biol. 15:1882-1888 or of HDL (Badimon et al, 1989, Lab Invest. 60:455-461 and J Clin Invest. 85:1234-1241, 1990) in experimental animals and initial human clinical studies (Nanjee et al., 1999, Arterioscler Thromb Vasc Biol. 19:979-989 and Eriksson et al. 1999, Circulation 100:594-598) has been shown to exert significant biochemical changes, as well as to reduce the extent and severity of atherosclerotic lesions.

Human gene therapy may provide a superior approach for achieving plaque reduction by providing prolonged and continuous expression of genes such as apoA-1 Milano. In the case of conventional gene therapy approaches that add back the entire apoA-1 cDNA, un-regulated expression of this cDNA may lead to toxicity. These problems could be overcome by utilization of spliceosome mediated RNA trans-splicing to convert the wild type apoA-1, or albumin, into Milano or other useful apoA-1 variants.

Similarly, spliceosome mediated RNA trans-splicing may be used to simultaneously reduce the expression of apoB, a major component of low-density lipoprotein, and produce HDL, i.e., express apoA-1 wild type or the Milano variant or convert other expressed proteins such as albumin to produce ApoA-1-Milano function.

### SUMMARY OF THE INVENTION

The present invention relates to compositions and methods for generating novel nucleic acid molecules through spliceosome-mediated targeted RNA trans-splicing, ribozyme mediated trans-splicing, or other means of converting mRNA. The compositions of the invention include pre-trans-splicing molecules (hereinafter referred to as “PTMs”) designed to interact with a natural target pre-mRNA molecule (hereinafter referred to as “pre-mRNA”) and mediate a spliceosomal trans-splicing reaction resulting in the generation of a novel chimeric RNA molecule (hereinafter referred to as “chimeric RNA”). The methods of the invention encompass contacting the PTMs of the invention with a natural target pre-mRNA under conditions in which a portion of the PTM is spliced to the natural pre-mRNA to form a novel chimeric RNA. The PTMs of the invention are genetically engineered so that the novel chimeric RNA resulting from the trans-splicing reaction may encode a protein that provides health benefits. Generally, the target pre-mRNA is chosen because it is expressed within a specific cell type thereby providing a means for targeting expression of the novel chimeric RNA to a selected cell type. For example, PTMs may be targeted to pre-mRNAs expressed in the liver such as apoA-1 and/or albumin pre-mRNA.

In particular, the compositions of the invention include pre-trans-splicing molecules (hereinafter referred to as “PTMs”) designed to interact with an apoA-1 target pre-mRNA molecule (hereinafter referred to as “apoA-1 pre-mRNA”) and mediate a spliceosomal trans-splicing reaction resulting in the generation of a novel chimeric RNA molecule (hereinafter referred to as “chimeric RNA”).

The compositions of the invention further include PTMs designed to interact with albumin target pre-mRNA molecule (hereinafter referred to as “albumin pre-mRNA”) and mediate a spliceosomal trans-splicing reaction resulting in the generation of a novel chimeric RNA molecule.

The compositions of the invention further include PTMs designed to interact with an apoB target pre-mRNA molecule (hereinafter referred to as “apoB pre-mRNA”) and mediate a spliceosomal trans-splicing reaction resulting in the generation of a novel chimeric RNA molecule.

The compositions of the invention include PTMs designed to interact with an apoA-1 target pre-mRNA molecule, albumin target pre-mRNA, or an apoB target pre-mRNA or other pre-mRNA targets and mediate a spliceosomal trans-splicing reaction resulting in the generation of a novel chimeric RNA molecule. Such PTMs are designed to produce an apoA-1 or other apoA-1 variants including Milano which are useful to protect against atherosclerosis.

The general design, construction and genetic engineering of PTMs and demonstration of their ability to successfully mediate trans-splicing reactions within the cell are described in detail in U.S. Pat. Nos. 6,083,702, 6,013,487 and 6,280,978 as well as patent Ser. Nos. 09/756,095, 09/756,096, 09/756,097 and 09/941,492, the disclosures of which are incorporated by reference in their entirety herein.

The general design, construction and genetic engineering of trans-splicing ribozymes and demonstration of their ability to successfully mediate trans-splicing reactions within the cell are described in detail in and U.S. Pat. Nos. 5,667,969, 5,854,038 and 5,869,254, as well as patent Serial No. 20030036517, the disclosures of which are incorporated by reference in their entirety herein.

The methods of the invention encompass contacting the PTMs of the invention with an apoA-1 target pre-mRNA, albumin target pre-mRNA, or apoB target pre-mRNA, or other expressed pre-mRNA targets, under conditions in which a portion of the PTM is spliced to the target pre-mRNA to form a novel chimeric RNA. The methods of the invention comprise contacting the PTMs of the invention with a cell expressing an apoA-1 target pre-mRNA, or an apoB target pre-mRNA or other expressed pre-mRNA targets, such as albumin pre-mRNA, under conditions in which the PTM is taken up by the cell and a portion of the PTM is trans-spliced to a portion of the target pre-mRNA to form a novel chimeric RNA molecule that results in expression of the an apoA-1 Milano or another variant. Alternatively, for example, when targeting the albumin or apoB pre-mRNAs, the novel chimeric RNA may encode a wild type apoA-1 protein.

Alternatively, nucleic acid molecules encoding the PTMs of the invention may be delivered into a target cell followed by expression of the nucleic acid molecule to form a PTM capable of mediating a trans-splicing reaction. The PTMs of the invention are genetically engineered so that the novel chimeric RNA resulting from the trans-splicing reaction may encode the apoA-1 Milano variant protein which has been shown to reduce plaque buildup which may be useful in the prevention or treatment of vascular disease. Alternatively, the chimeric mRNA may encode a wild type apoA-1 protein. Thus, the methods and compositions of the invention can be used in gene therapy for the prevention and treatment of vascular disorders resulting from accumulation of plaque which is a risk factor associated with heart attacks and strokes.

### BRIEF DESCRIPTION OF THE DRAWINGS

FIG. 1. Schematic representation of different trans-splicing reactions. (a) trans-splicing reactions between the target 5′ splice site and PTM's 3′ splice site, (b) trans-splicing reactions between the target 3′ splice site and PTM's 5′ splice site and (c) replacement of an internal exon by a double trans-splicing reaction in which the PTM carries both 3′ and 5′ splice sites. BD, binding domain; BP, branch point sequence; PPT, polypyrimidine tract; and ss, splice sites.

FIG. 2. Human ApoA-1 gene and mRNA. The ApoA-1 gene is 1.87 kb long and comprises 4 exons including a non-coding exon 1. The apoA-1 mRNA is 897 nucleotides long including a 5′ UTR and 3′UTR. The apoA-1 amino acid sequence consists of 267 residues including a 24 amino acid signal peptide at the N-terminus and the mature protein is a single polypeptide chain with 243 amino acid residues.

FIG. 3A. Nucleotide and amino acid sequence of wild type ApoA-1. FIG. 3B. ApoA-1-Milano variant.

FIG. 3C. Strategy to create ApoA-1-Milano.

FIG. 4. Target gene and PTM structure. FIG. 4A. Schematic structure of human wild type apoA-1 full length target gene for in vitro studies. FIG. 4B Schematic structure of human apoA-1 Milano PTM1 (exon 4).

FIG. 5. Schematic illustration of trans-splicing reaction between apoA-1 target pre mRNA and PTM.

FIG. 6. ApoB-100 gene and mRNA.

FIG. 7. Schematic structure of ApoB target pre-mRNA.

FIG. 8. Mini-gene target and PTM structure.

FIG. 8A. Schematic structure of human apoB mini-gene target for in vitro studies.

FIG. 8B. Schematic structure of human apoA-1 Milano PTM2.

FIG. 9. Schematic illustration of trans-splicing reaction between apoB target pre mRNA and PTM).

FIG. 10. Human Albumin Gene Structure. (See, also Minghetti et al., 1986, J. Biol. Chem. 261:6747-6757).

FIG. 11. Human ApoA-1.

FIG. 12. Human ApoA-1 Gene and mRNA structural details

FIG. 13. Schematic illustration of human and mouse albumin exon 1/human ApoA-1 fusions.

FIG. 14. Nucleotide sequences of human albumin exon 1/human ApoA-1 (wild type) fusion. Underlined sequence represents human albumin signal peptide; / indicates fusion junction between albumin and ApoA-1. ATG and stop codon, TGA are indicated in italics.

FIG. 15. Western Anaysis of Mouse and Human Alb/ApoA-1 Fusion in 293 cells.

FIG. 16. Western Anaysis of Mouse and Human Alb/ApoA-1 Fusion in 293 and HepG2 cells.

FIG. 17. Target Construct for Binding Domain Screen. Schematic structure of 5′ GFP-Albln1Ex2 target gene for in vitro studies. Target pre-mRNA construct contains partial coding sequence for GFP fluorescent protein followed by 5′ splice site, albumin intron 1, 3′ acceptor site and albumin exon 2.

FIG. 18. 5′ GFP-Albln1Ex2 Pre-mRNA Target Sequence. Nucleotide sequence of 5′ GFP-Albln1Ex2 gene for in vitro studies. Sequences shown in italics indicate first half of the coding sequence for GFP fluorescent protein followed by human albumin intron 1 and exon 2 sequences (underlined). “/” indicates 5′ and 3′ splice junctions.

FIG. 19. PTM Cassette Used for Binding Domain Screen. Schematic structure of a prototype PTM expression cassette is shown. It consists of a trans-splicing domain (TSD) followed by a 24 nucleotide spacer, a 3′ splice site including the consensus yeast branch point (BP), an extended polypyrimidine tract and the AG splice acceptor site. The TSD was fused to the remaining 3′GFP coding sequence. In addition, the PTM cassette also contain full length coding sequence for a second fluorescent reporter (DsRed2) and the expression is driven by an internal ribosome entry site (IRES) of the encephalomyocarditis virus (ECMV).

FIG. 20. Binding Domain Screening Strategy.

FIG. 21. Schematic of targeted trans-splicing of human ApoA-1 into albumin target pre-mRNA.

FIG. 22. Schematic of human and mouse Apo A-1 fusion constructs.

FIG. 23. SDS gels showing human Apo A-1 expression in 293 cells

FIG. 24. Western blot showing expression and secretion of mature human Apo A-1 protein in 293 cells

FIG. 25. Cholesterol efflux in 293 cells demonstrating the expression of functional human Apo A-1 protein.

FIG. 26A. Schematic of FACS-based PTM selection strategy.

FIG. 26B. Comparison of high capacity screening (HCS) protocols.

FIG. 27. Schematic of pre-mRNA target used in HCS.

FIG. 28. Schematic of PTM cassette used in HCS.

FIG. 29. PCR analysis of the mouse albumin binding domain (BD) library.

FIG. 30. High capacity screening (HCS) method and summary of results.

FIG. 31. Trans-splicing efficiency of PTMs selected from HCS.

FIG. 32. Bar graph showing trans-splicing efficiency and GFP fluorescence of various PTMs selected from HCS.

FIG. 33A. Schematic showing the relative position and sequences of mouse albumin lead binding domains (BDs) selected for functional studies.

FIG. 33B. Nucleotide sequences of binding domains selected from the HCS.

FIG. 34. Schematic showing the human Apo A-1 PTM expression cassette used for proof of principle in vitro studies.

FIG. 35. Schematic diagram of the mouse albumin mini-gene pre-mRNA target.

FIG. 36. Trans-splicing of mAlbPTMs into albumin exon 1 in stable cells.

FIG. 37. Western blot analysis of trans-spliced human Apo A-1 protein.

FIG. 38. PTM-mediated trans-splicing into endogenous albumin exon 1 in mice.

FIG. 39. Schematic diagram showing a human albumin targeting strategy to increase ApoA1 expression.

FIG. 40 Elimination of albumin sequence in the final trans-spliced product.

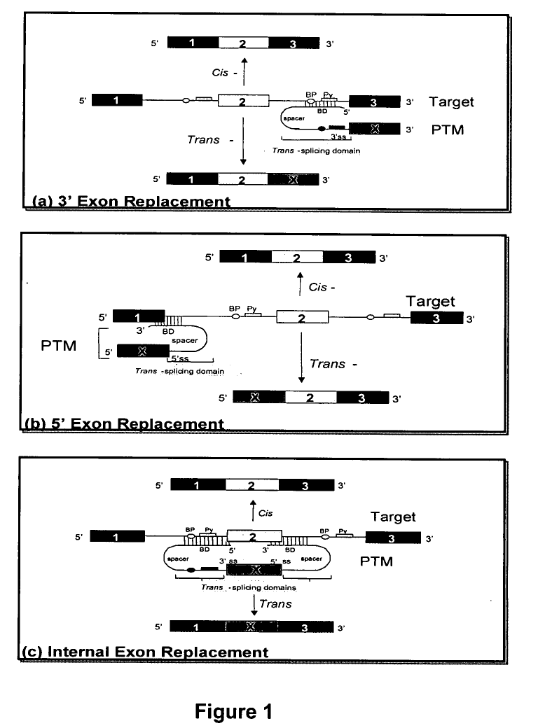

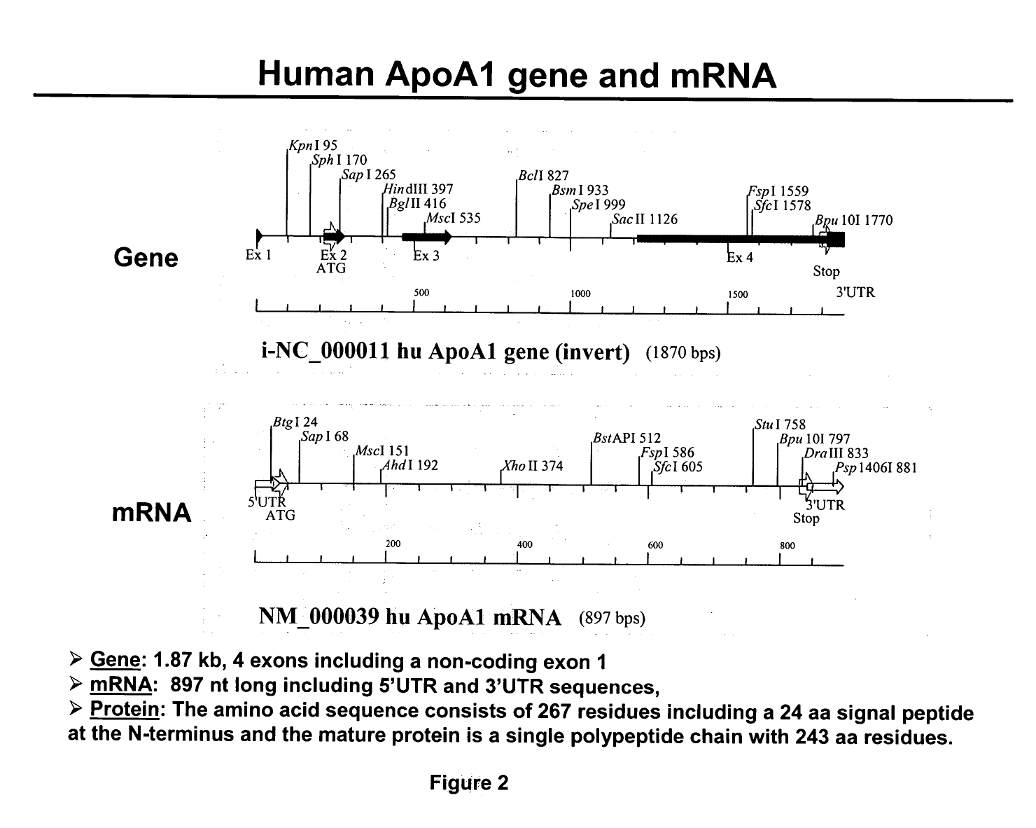

### DETAILED DESCRIPTION OF THE INVENTION

The present invention relates to novel compositions comprising pre-trans-splicing molecules (PTMs) and the use of such molecules for generating novel nucleic acid molecules. The PTMs of the invention comprise (i) one or more target binding domains that are designed to specifically bind to a apoA-1 or apoB target pre-mRNA or other expressed pre-mRNA targets, such as albumin pre-mRNA, (ii) a 3′ splice region that includes a branch point, pyrimidine tract and a 3′ splice acceptor site and/or a 5′ splice donor site; and (iii) additional nucleotide sequences such as those encoding for the the wild type apoA-1 or apoA-1 Milano variant. The PTMs of the invention may further comprise one or more spacer regions that separate the RNA splice site from the target binding domain.

The methods of the invention encompass contacting the PTMs of the invention with apoA-1 target pre-mRNA, or apoB target pre-mRNA, or other expressed pre-mRNA targets such as albumin target pre-mRNA, under conditions in which a portion of the PTM is trans-spliced to a portion of the target pre-mRNA to form a novel chimeric RNA that results in expression of the apoA-1 Milano variant, wild type apoA-1, or an apoB/apoA-1 Milano fusion protein, or other fusion protein encoding other variants of apoA-1.

### Structure of the Pre-Trans-Splicing Molecules

The present invention provides compositions for use in generating novel chimeric nucleic acid molecules through targeted trans-splicing. The PTMs of the invention comprise (i) one or more target binding domains that targets binding of the PTM to a apoA-1 or apoB pre-mRNA or other expressed pre-mRNA targets such as, for example, albumin pre-mRNA (ii) a 3′ splice region that includes a branch point, pyrimidine tract and a 3′ splice acceptor site and/or 5′ splice donor site; and (iii) coding sequences for apoA-1 Milano, other variants of apoA-1 or wild type apoA-1. The PTMs of the invention may also include at least one of the following features: (a) binding domains targeted to intron sequences in close proximity to the 3′ or 5′ splice signals of the target intron, (b) mini introns, (c) ISAR (intronic splicing activator and repressor)-like cis-acting elements, and/or (d) ribozyme sequences. The PTMs of the invention may further comprise one or more spacer regions to separate the RNA splice site from the target binding domain.

The general design, construction and genetic engineering of such PTMs and demonstration of their ability to mediate successful trans-splicing reactions within the cell are described in detail in U.S. Pat. Nos. 6,083,702, 6,013,487 and 6,280,978 as well as patent Ser. Nos. 09/941,492, 09/756,095, 09/756,096 and 09/756,097 the disclosures of which are incorporated by reference in their entirety herein.

The target binding domain of the PTM endows the PTM with a binding affinity for the target pre-mRNA, i.e., an apoA-1 or apoB target pre-mRNA, or other pre-mRNA targets such as, for example, albumin pre-mRNA. As used herein, a target binding domain is defined as any molecule, i.e., nucleotide, protein, chemical compound, etc., that confers specificity of binding and anchors the pre-mRNA closely in space to the PTM so that the spliceosome processing machinery of the nucleus can trans-splice a portion of the PTM to a portion of the pre-mRNA. The target pre-mRNA may be mammalian, such as but not limited to, mouse, rat, bovine, goat, or human pre-RNA.

The target binding domain of the PTM may contain multiple binding domains which are complementary to and in anti-sense orientation to the targeted region of the selected pre-mRNA, i.e., an apoA-1, apoB or albumin target pre-mRNA. The target binding domains may comprise up to several thousand nucleotides. In preferred embodiments of the invention the binding domains may comprise at least 10 to 30 and up to several hundred or more nucleotides. The efficiency and/or specificity of the PTM may be increased significantly by increasing the length of the target binding domain. For example, the target binding domain may comprise several hundred nucleotides or more. In addition, although the target binding domain may be “linear” it is understood that the RNA will very likely fold to form a secondary “safety” structure that may sequester the PTM splice site(s) until the PTM encounters it's pre-mRNA target, thereby increasing the specificity of trans-splicing. A second target binding region may be placed at the 3′ end of the molecule and can be incorporated into the PTM of the invention. Absolute complementarily, although preferred, is not required. A sequence “complementary” to a portion of an RNA, as referred to herein, means a sequence having sufficient complementarity to be able to hybridize with the target pre-mRNA, forming a stable duplex. The ability to hybridize will depend on both the degree of complementarity and the length of the nucleic acid (see, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch or length of duplex by use of standard procedures to determine the stability of the hybridized complex.

Binding may also be achieved through other mechanisms, for example, through triple helix formation, aptamer interactions, antibody interactions or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

In a specific embodiment of the invention, the target binding domain is complementary and in anti-sense orientation to sequences of the apoA-1, apoB, or albumin target pre-mRNA, which hold the PTM in close proximity to the target for trans-splicing. For example, a target binding domain may be defined as any molecule, i.e., nucleotide, protein, chemical compound, etc., that confers specificity of binding and anchors the apoA-1, or apoB or albumin pre-mRNA closely in space to the PTM so that the spliceosome processing machinery of the nucleus can trans-splice a portion of the PTM to a portion of the apoA-1, or apoB, or albumin pre-mRNA.

The PTM molecule also contains a 3′ splice region that includes a branchpoint sequence and a 3′ splice acceptor AG site and/or a 5′ splice donor site. The 3′ splice region may further comprise a polypyrimidine tract. Consensus sequences for the 5′ splice donor site and the 3′ splice region used in RNA splicing are well known in the art (see, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and /=the splice site). The 3′ splice site consists of three separate sequence elements: the branchpoint or branch site, a polypyrimidine tract and the 3′ consensus sequence (YAG). The branch point consensus sequence in mammals is YNYURAC (Y=pyrimidine; N=any nucleotide). The underlined A is the site of branch formation. A polypyrimidine tract is located between the branch point and the splice site acceptor and is important for efficient branch point utilization and 3′ splice site recognition. Recently, pre-mRNA introns beginning with the dinucleotide AU and ending with the dinucleotide AC have been identified and referred to as U12 introns. U12 intron sequences as well as any sequences that function as splice acceptor/donor sequences may also be used to generate the PTMs of the invention.

A spacer region to separate the RNA splice site from the target binding domain may also be included in the PTM. The spacer region may be designed to include features such as (i) stop codons which would function to block translation of any unspliced PTM and/or (ii) sequences that enhance trans-splicing to the target pre-mRNA.

In a preferred embodiment of the invention, a “safety” is also incorporated into the spacer, binding domain, or elsewhere in the PTM to prevent non-specific trans-splicing (Puttaraju et al., 1999 Nat. Boiotech, 17:246-252; Mansfield S G et al., 2000, Gene therapy, 7:1885-1895). This is a region of the PTM that covers elements of the 3′ and/or 5′ splice site of the PTM by relatively weak complementarity, preventing non-specific trans-splicing. The PTM is designed in such a way that upon hybridization of the binding/targeting portion(s) of the PTM, the 3′ and/or 5′ splice site is uncovered and becomes fully active.

Such “safety” sequences comprise one or more complementary stretches of cis-sequence (or could be a second, separate, strand of nucleic acid) which binds to one or both sides of the PTM branch point, pyrimidine tract, 3′ splice site and/or 5′ splice site (splicing elements), or could bind to parts of the splicing elements themselves. This “safety” binding prevents the splicing elements from being active (i.e. block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

Nucleotide sequence encoding for exon 4, exons 3-4, or exons 2-4 of the apoA-1 Milano variant are also included in the PTM of the invention. For example, the nucleotide sequence can include those sequences encoding gene products missing or altered in known genetic diseases. In addition, nucleotide sequences encoding marker proteins or peptides which may be used to identify or image cells may be included in the PTMs of the invention. In yet another embodiment of the invention nucleotide sequences encoding affinity tags such as, HIS tags (6 consecutive histidine residues) (Janknecht, et al., 1991, Proc. Natl. Acad. Sci. USA 88:8972-8976), the C-terminus of glutathione-S-transferase (GST) (Smith and Johnson, 1986, Proc. Natl. Acad. Sci. USA 83:8703-8707) (Pharmacia), FLAG (Asp-Tyr-Lys-Asp-Asp-Asp-Asp-Lys) (Eastman Kodak/IBI, Rochester, N.Y.), or CDC2 PSTAIRE epitope tag can be included in PTM molecules for use in affinity purification.

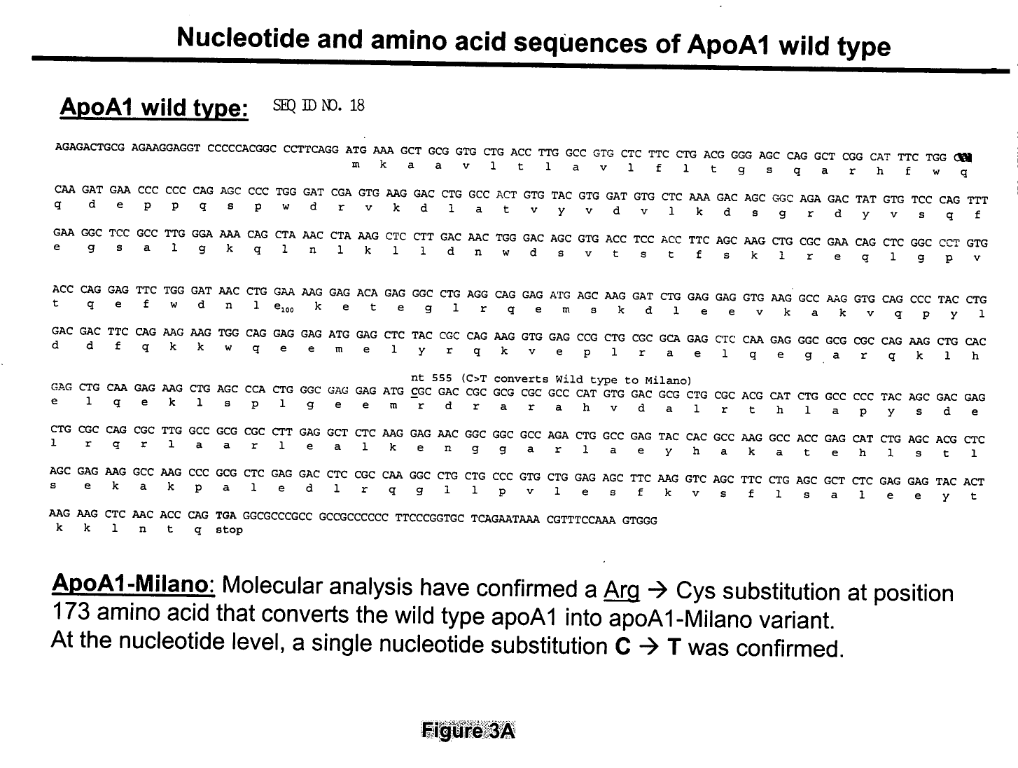

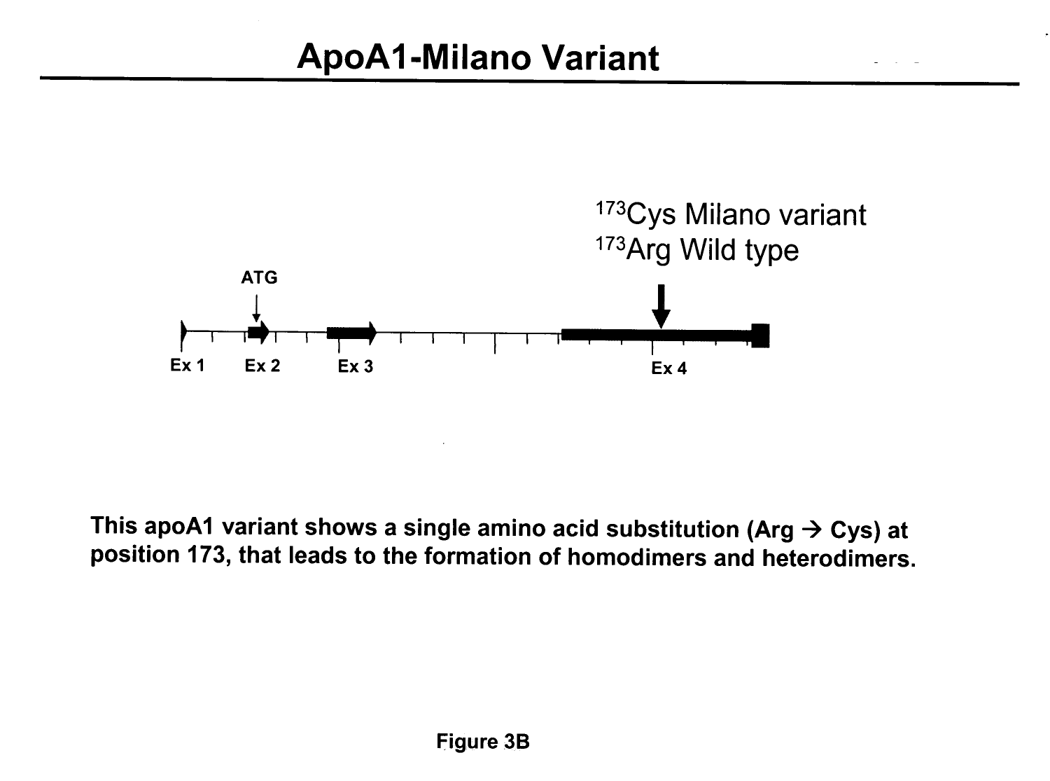

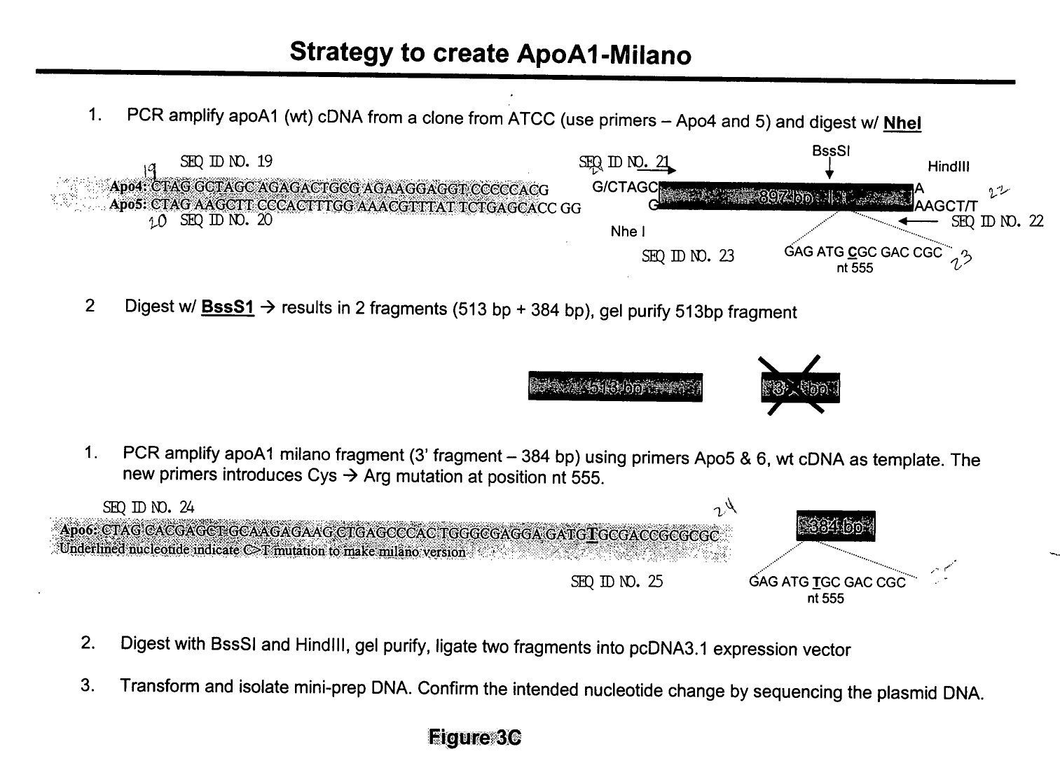

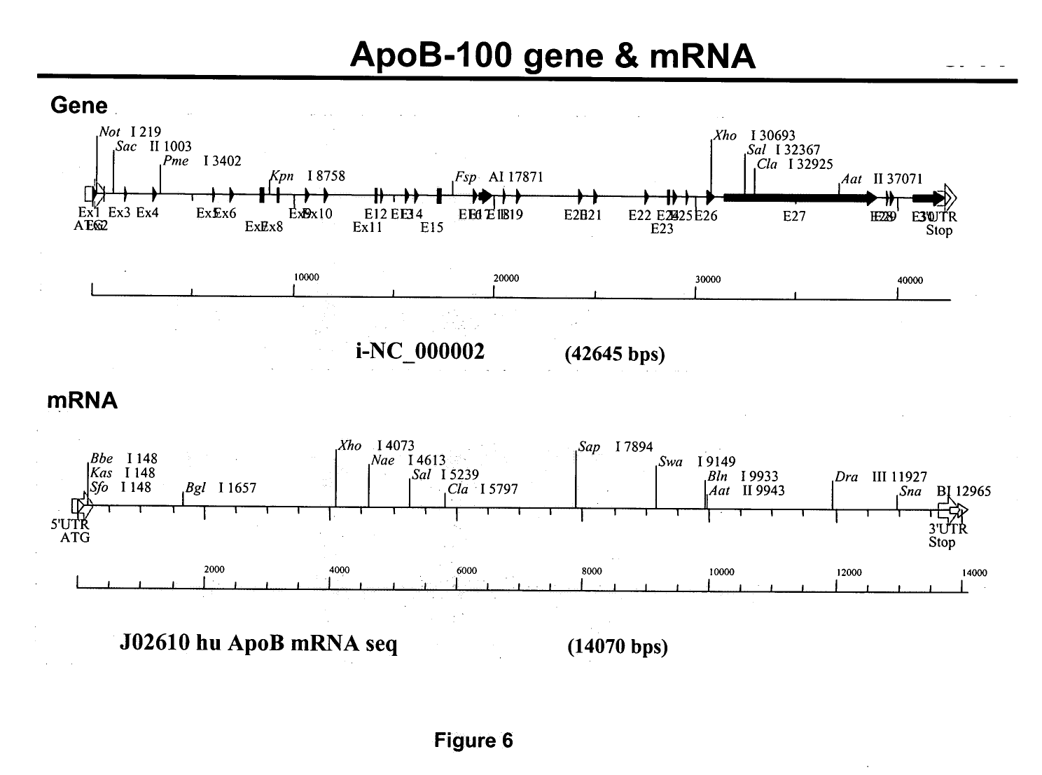

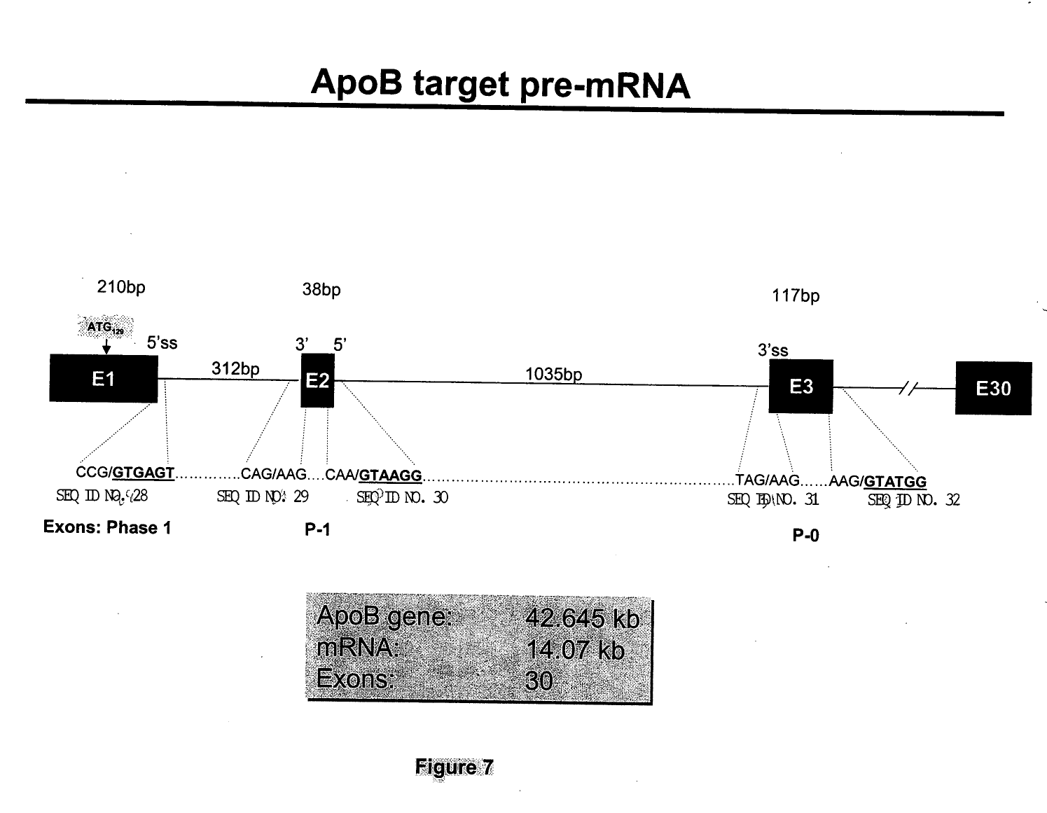

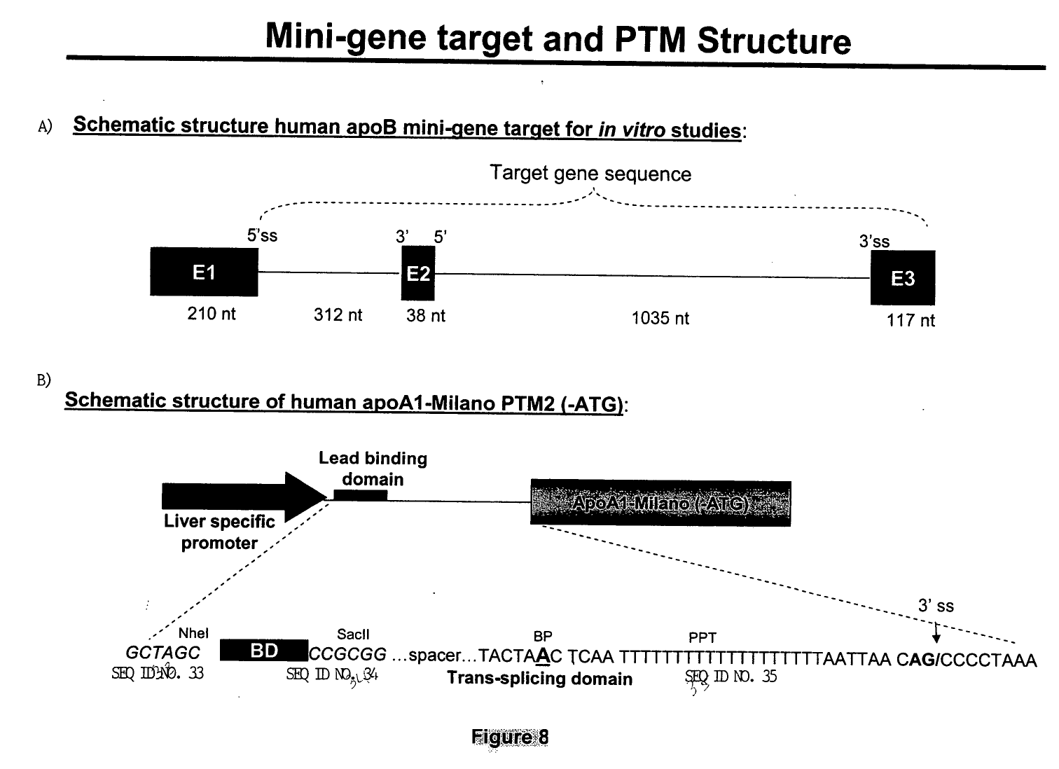

In a preferred embodiment of the invention, the PTMs of the invention contain apoA-1 exon 4 with an Arg to Cys substitution at position 173 (hereinafter referred to as “Arg→Cys”), thereby leading to the expression of apoA-1 Milano variant protein. A variety of different PTM molecules may be synthesized to substitute (Arg→Cys) at position 173. The PTMs of the invention may contain apoA-1 exon or exons, which when trans-spliced to the apoA-1, or apoB, target pre-mRNA or other pre-mRNA targets, will result in the formation of a composite or chimeric RNA capable of encoding an apoA-1 Milano variant protein, or an apoB/apoA-1 Milano variant protein. The nucleotide sequence of the apoA-1 gene is known, as well as the mutation leading to expression of the Milano variant and incorporated herein in its entirety (FIG. 3A-B). Likewise, the nucleotide sequence of the apoB gene is known (FIG. 6).

The apoA-1 exon sequences to be included in the structure of the PTM will be designed to include apoA-1 exon 4 sequences as depicted in FIG. 4. In such an instance, 3′ exon replacement will result in the formation of a chimeric RNA molecule that encodes for apoA-1 Milano variant protein having a Arg→Cys substitution at position 173.

The PTM's of the invention may be engineered to contain a single apoA-1 exon sequence, multiple apoA-1 exon sequences, or alternatively the complete set of 4 exon sequences. The number and identity of the apoA-1 sequences to be used in the PTMs will depend on the type of trans-splicing reaction, i.e., 5′ exon replacement, 3′ exon replacement or internal exon replacement, as well as the pre-mRNA targets.

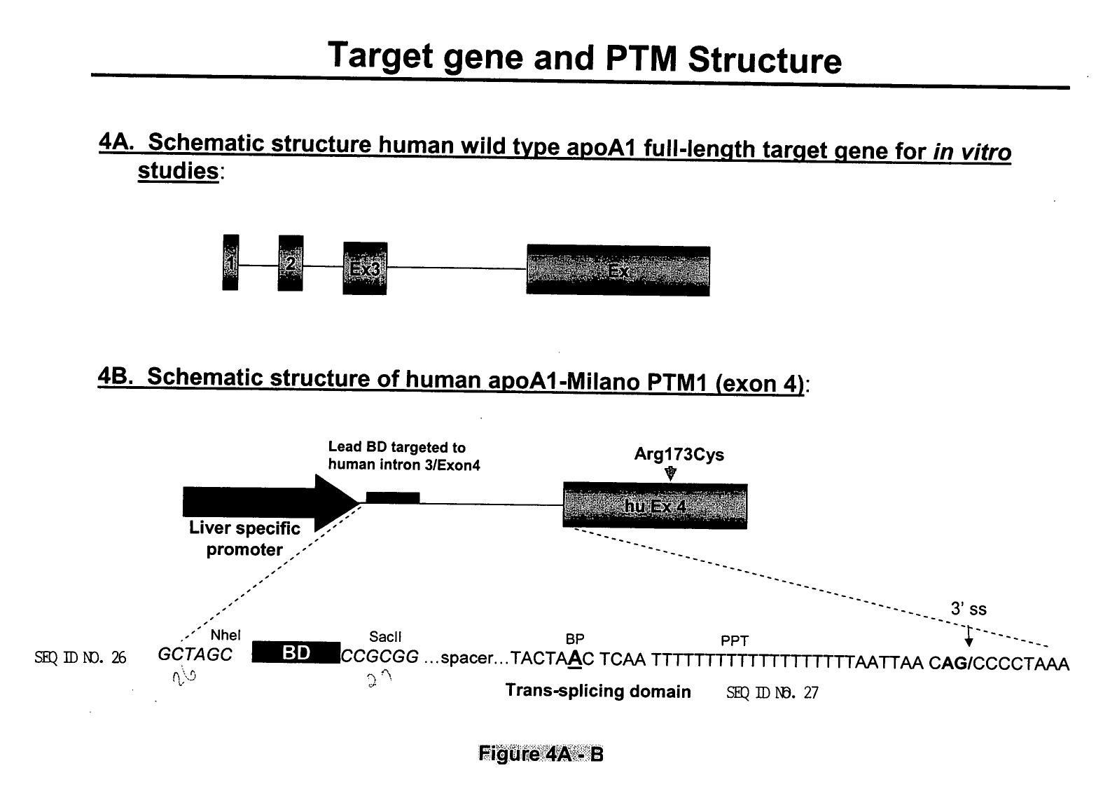

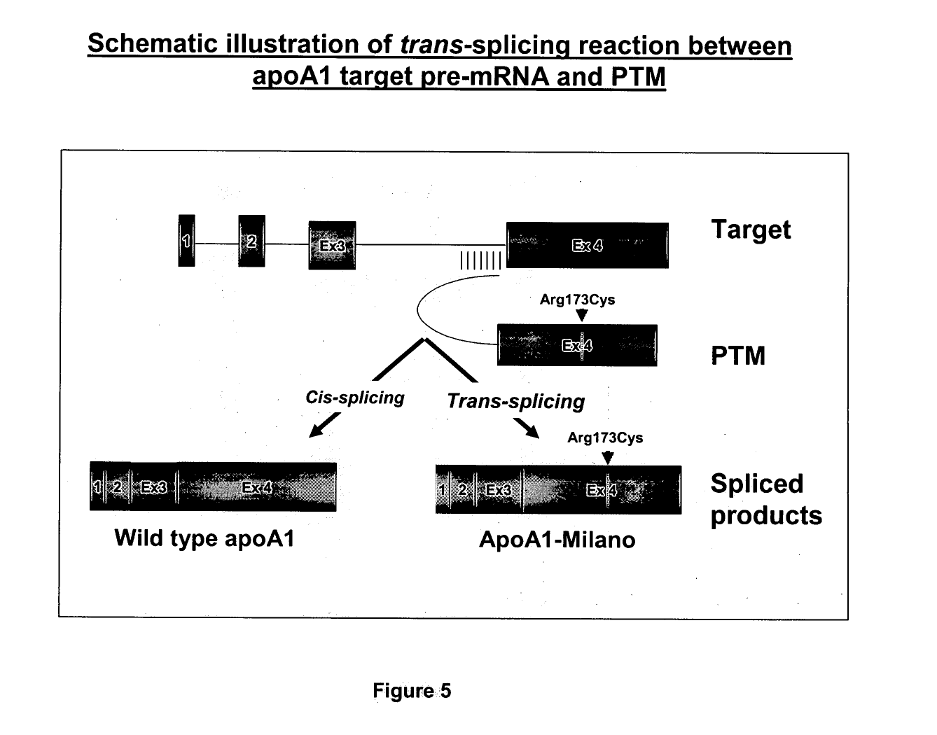

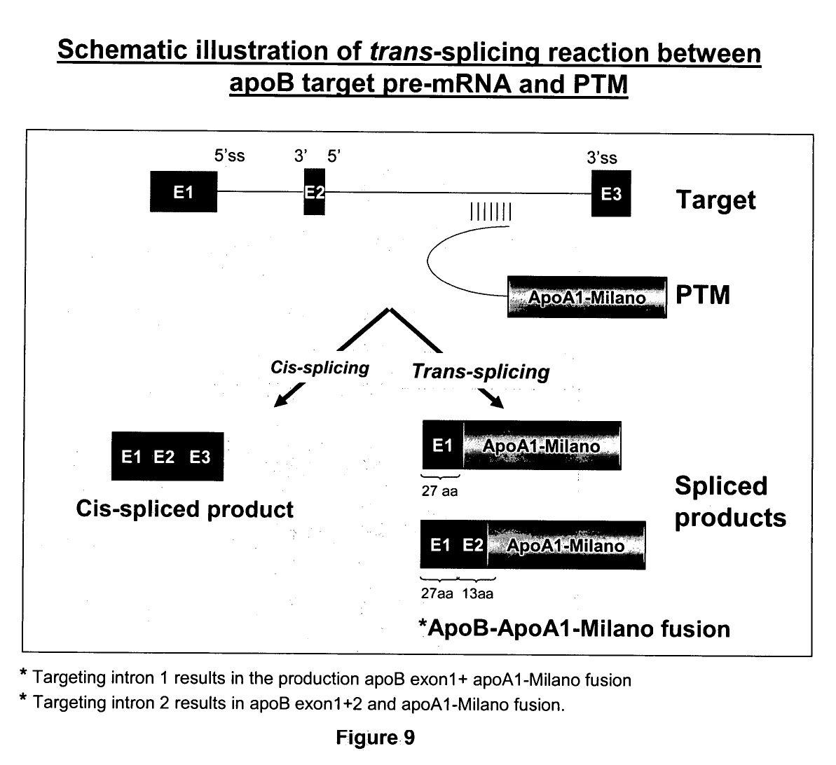

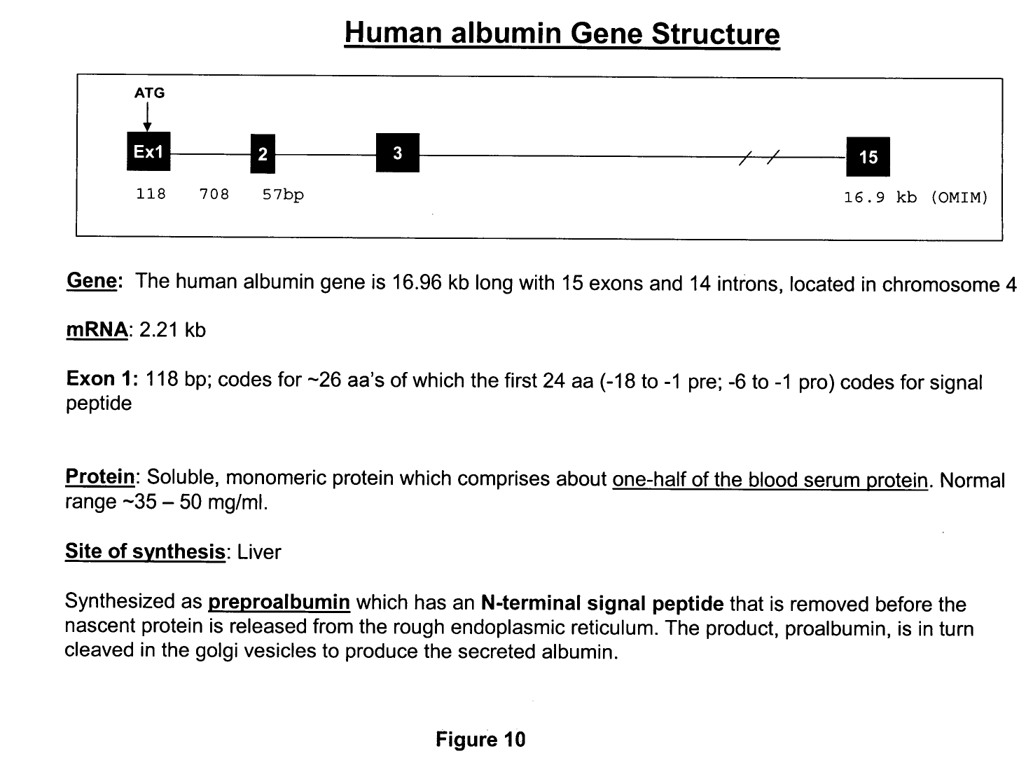

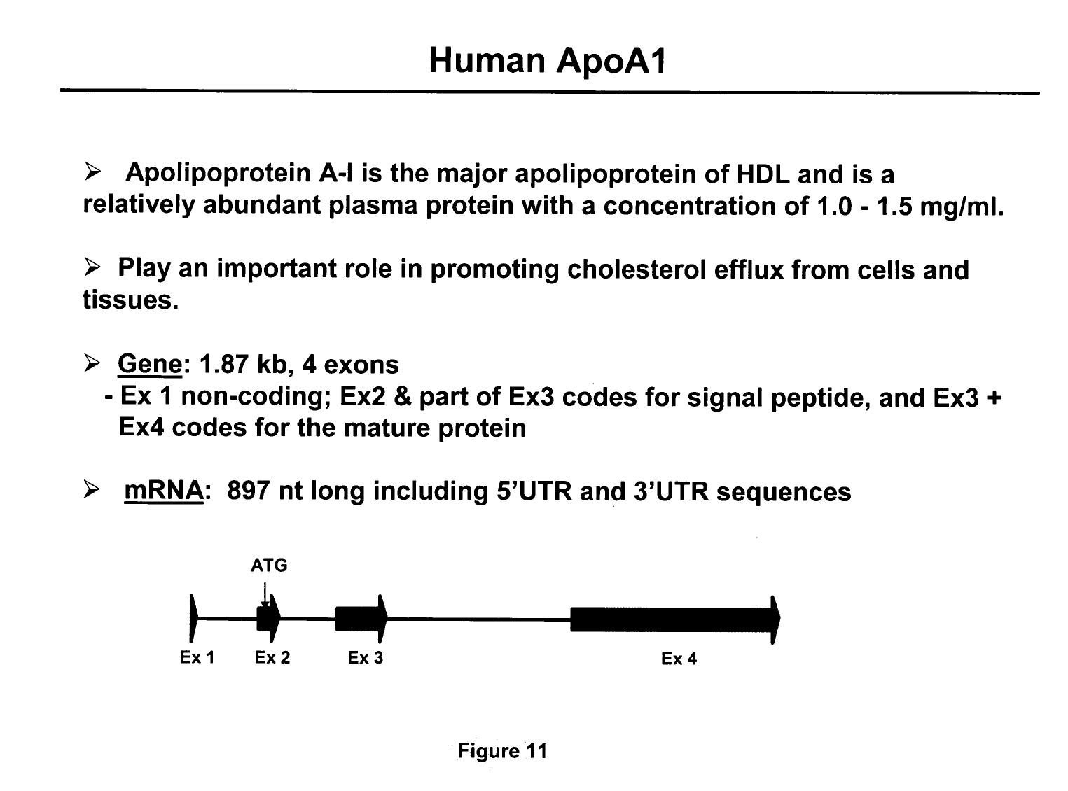

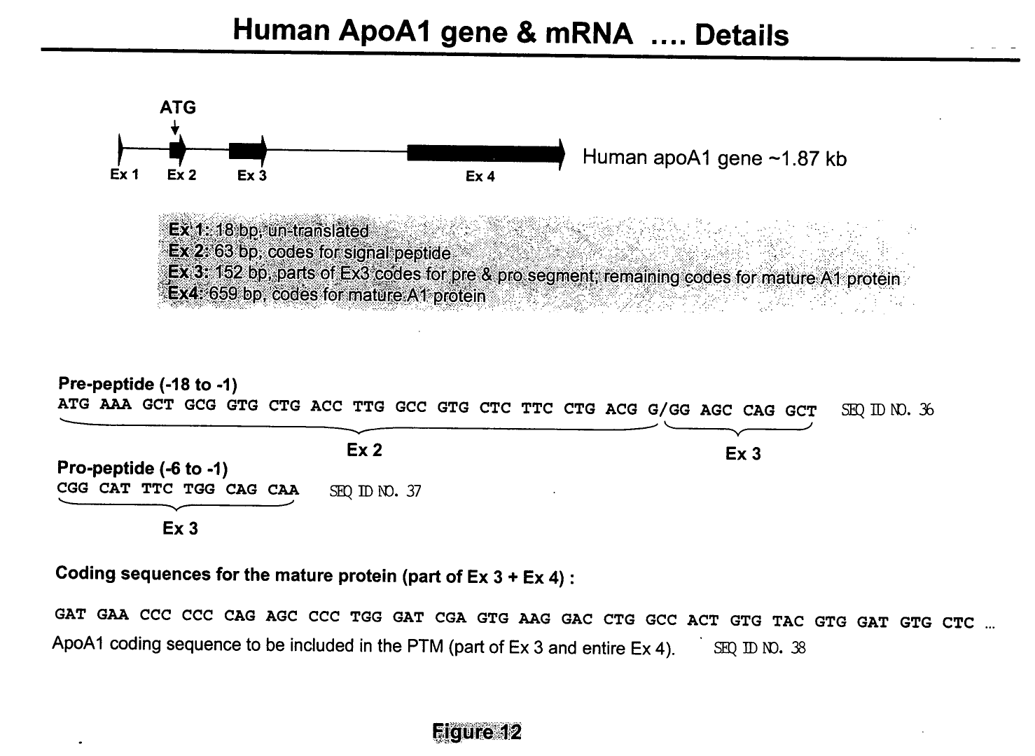

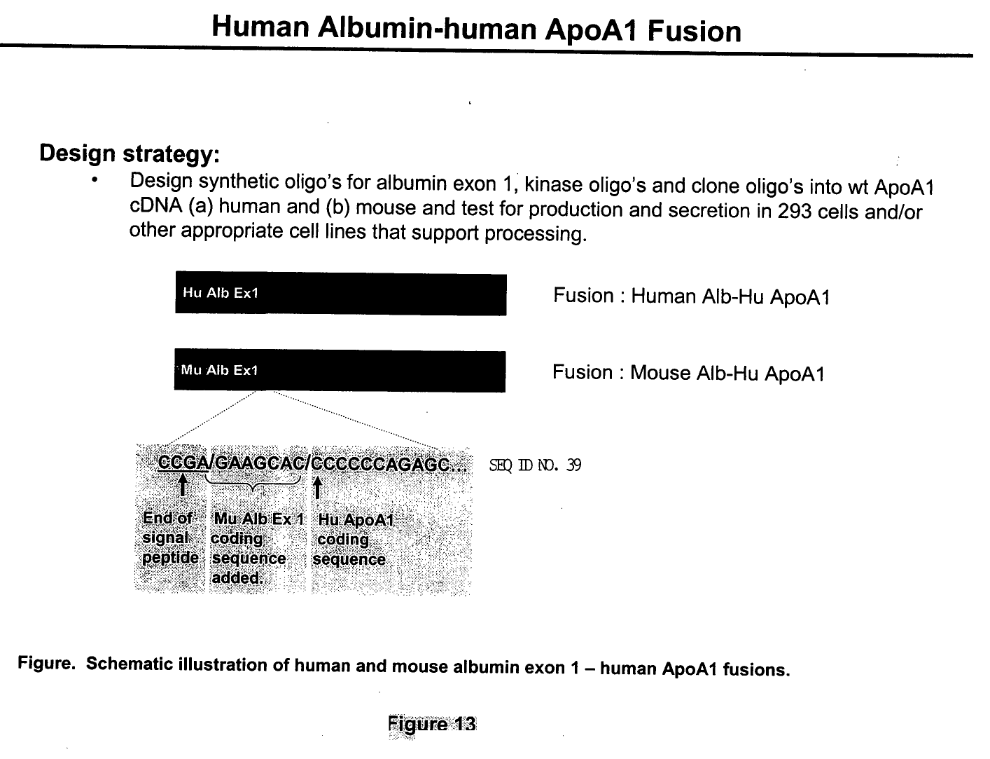

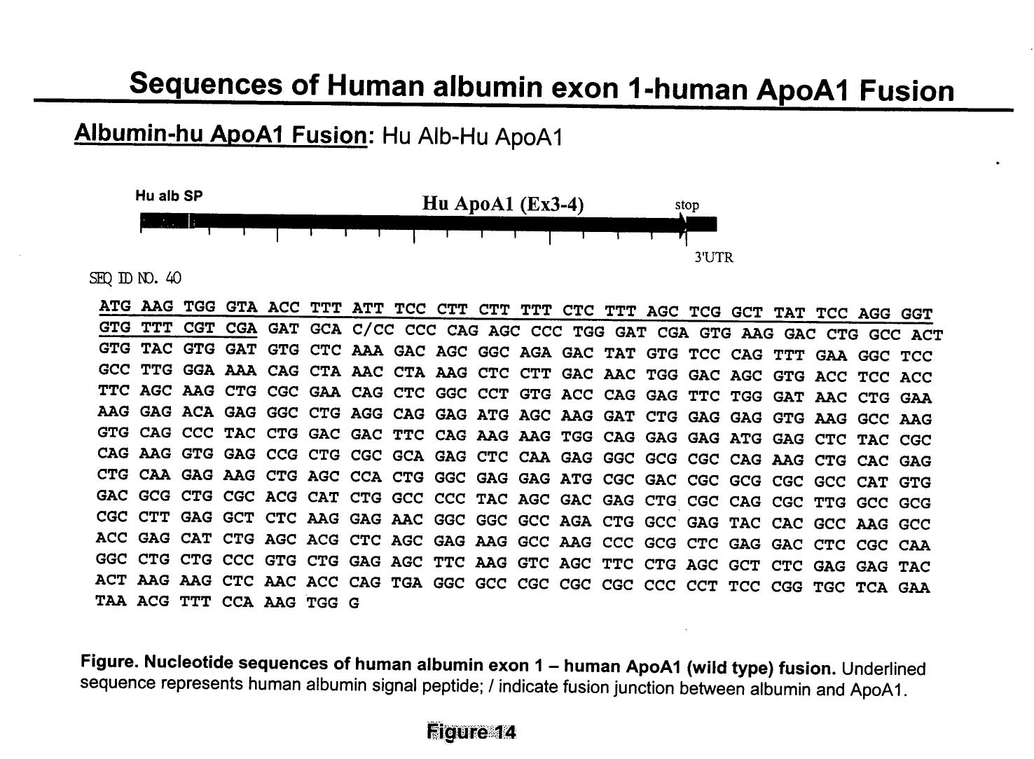

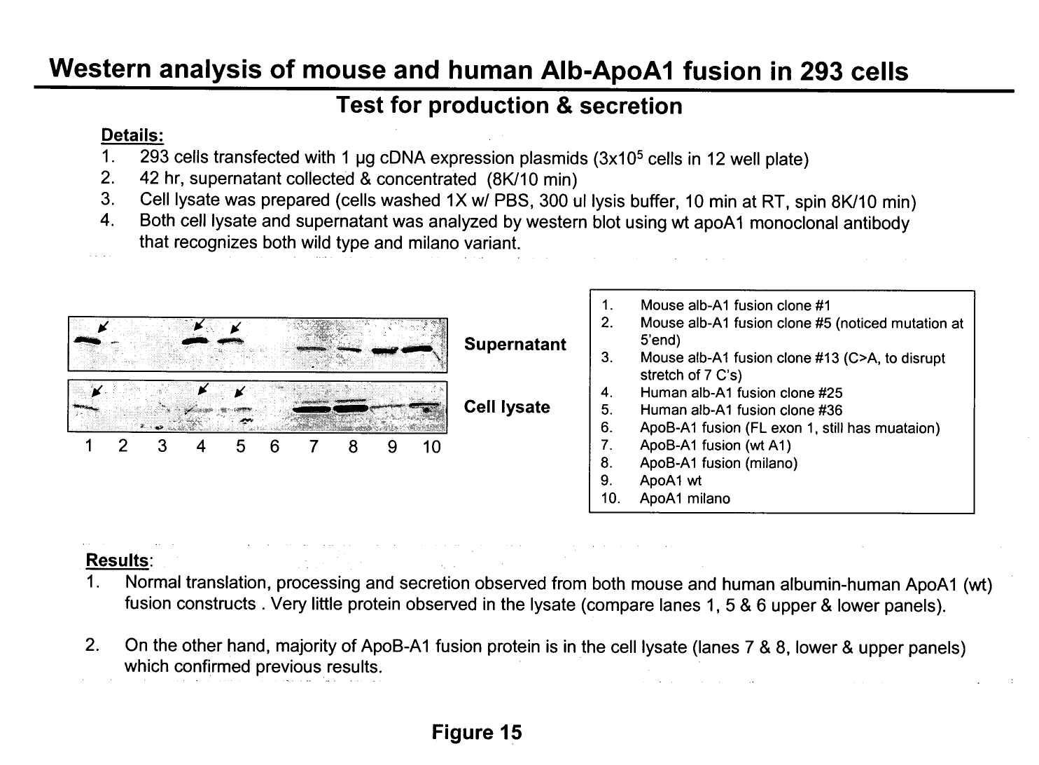

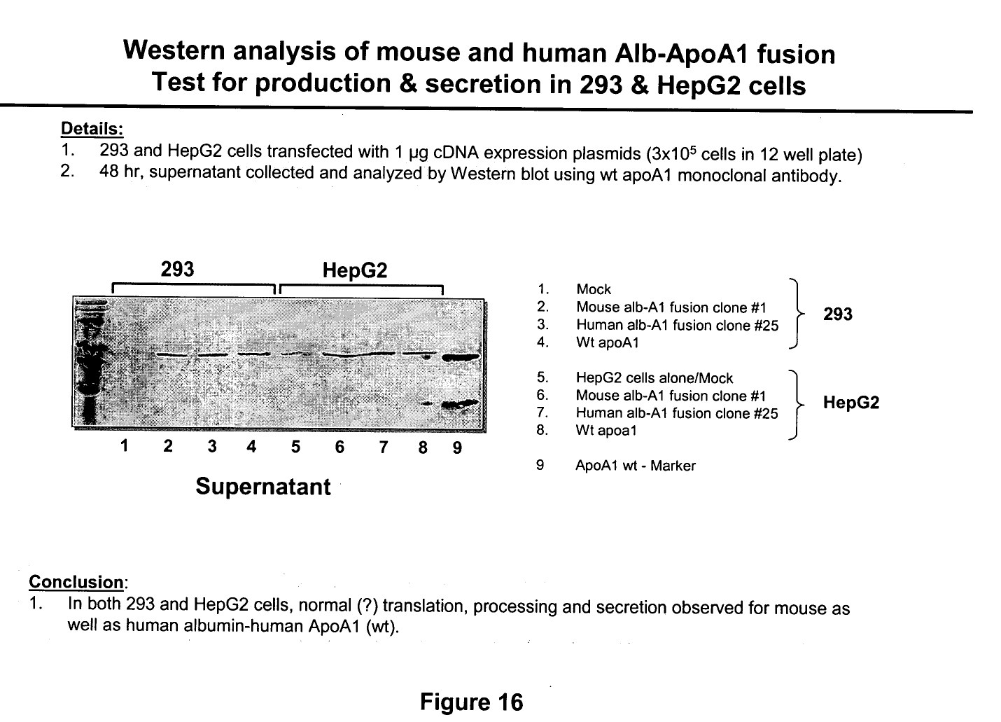

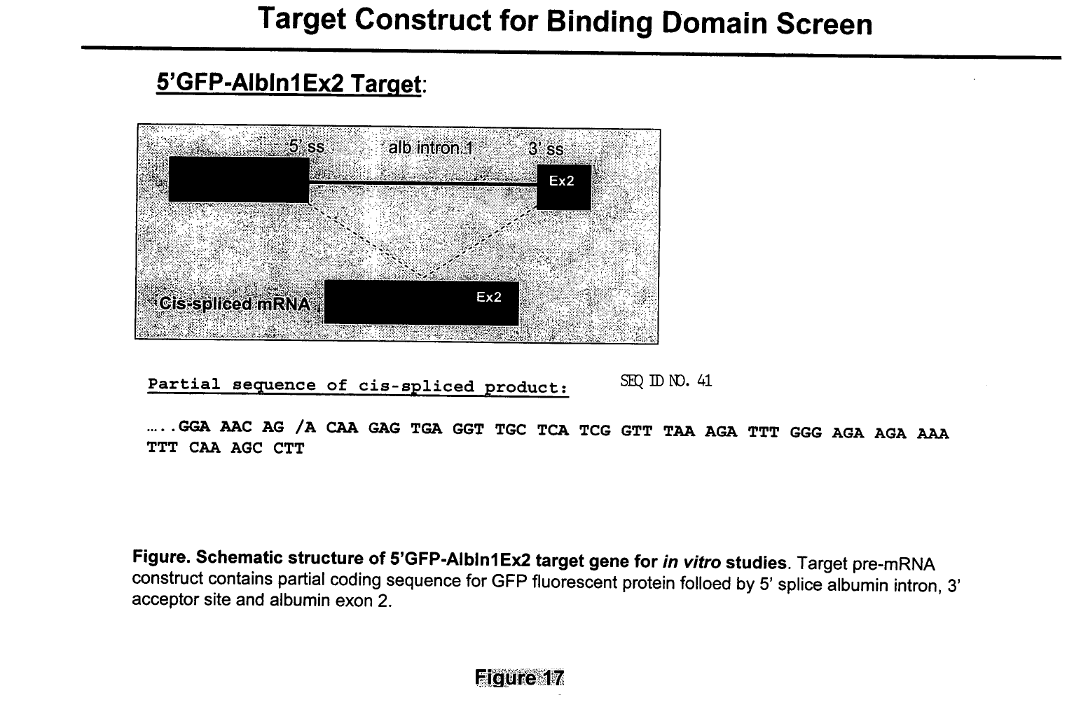

Specific PTMs of the invention, include but are not limited to, those containing nucleic acids encoding apoA-1 exon 4 sequences. Such PTMs may be used for mediating a 3′ exon replacement trans-splicing reaction as depicted in FIGS. 5, 9 and 21.

Specific PTMs of the invention include, but are not limited to, those containing nucleic acid sequences encoding apoA-1-Milano. Such PTMs may be used for mediating a 5′ exon replacement trans-splicing reaction. These PTMs would contain the N-terminal portion of the coding sequence, including the Milano mutation. In addition, PTMs of the invention may comprise a single apoA-1 variant exon or any combination of two or more apoA-1 variant exons.

Further, the PTMs of the invention include, but are not limited to, those containing nucleic acid sequences encoding wild type ApoA-1.

The present invention further provides PTM molecules wherein the coding region of the PTM is engineered to contain mini-introns. The insertion of mini-introns into the coding sequence of the PTM is designed to increase definition of the exon and enhance recognition of the PTM splice sites. Mini-intron sequences to be inserted into the coding regions of the PTM include small naturally occurring introns or, alternatively, any intron sequences, including synthetic mini-introns, which include 5′ consensus donor sites and 3′ consensus sequences which include a branch point, a 3′ splice site and in some instances a pyrimidine tract.

The mini-intron sequences are preferably between about 60-150 nucleotides in length, however, mini-intron sequences of increased lengths may also be used. In a preferred embodiment of the invention, the mini-intron comprises the 5′ and 3′ end of an endogenous intron. In preferred embodiments of the invention the 5′ intron fragment is about 20 nucleotides in length and the 3′ end is about 40 nucleotides in length.

In a specific embodiment of the invention, an intron of 528 nucleotides comprising the following sequences may be utilized. Sequence of the intron construct is as follows:

5′ fragment sequence:

Gtagttcttttgttcttcactattaagaacttaatttggtgtccatgtctctttttttttctagtttgtagtgctggaaggtatttttggaga
aattcttacatgagcattaggagaatgtatgggtgtagtgtcttgtataatagaaattgttccactgataatttactctagttttttatttcctcatattat
tttcagtggctttttcttccacatctttatattttgcaccacattcaacactgtagcggccgc.

3′ fragment sequence:

Ccaactatctgaatcatgtgccccttctctgtgaacctctatcataatacttgtcacactgtattgtaattgtctcttttactttcccttg
tatcttttgtgcatagcagagtacctgaaacaggaagtattttaaatattttgaatcaaatgagttaatagaatctttacaaataagaatatacactt
ctgcttaggatgataattggaggcaagtgaatcctgagcgtgatttgataatgacctaataatgatgggttttatttccag.

In an embodiment of the invention the Tia-1 binding sequences are inserted within 100 nucleotides from the 5′ donor site (Del GAtto-Konczak et al., 2000, Mol. Cell Biol. 20:6287-6299). In a preferred embodiment of the invention the Tia-1 binding sequences are inserted within 50 nucleotides from the 5′ donor site. In a more preferred embodiment of the invention the Tia-1 sequences are inserted within 20 nucleotides of the 5′ donor site.

The compositions of the invention further comprise PTMs that have been engineered to include cis-acting ribozyme sequences. The inclusion of such sequences is designed to reduce PTM translation in the absence of trans-splicing or to produce a PTM with a specific length or defined end(s). The ribozyme sequences that may be inserted into the PTMs include any sequences that are capable of mediating a cis-acting (self-cleaving) RNA splicing reaction. Such ribozymes include but are not limited to hammerhead, hairpin and hepatitis delta virus ribozymes (see, Chow et al. 1994, J Biol Chem 269:25856-64).

In an embodiment of the invention, splicing enhancers such as, for example, sequences referred to as exonic splicing enhancers may also be included in the PTM design. Transacting splicing factors, namely the serine/arginine-rich (SR) proteins, have been shown to interact with such exonic splicing enhancers and modulate splicing (see, Tacke et al., 1999, Curr. Opin. Cell Biol. 11:358-362; Tian et al., 2001, J Biological Chemistry 276:33833-33839; Fu, 1995, RNA 1:663-680). Nuclear localization signals may also be included in the PTM molecule (Dingwell and Laskey, 1986, Ann. Rev. Cell Biol. 2:367-390; Dingwell and Laskey, 1991, Trends in Biochem. Sci. 16:478-481). Such nuclear localization signals can be used to enhance the transport of synthetic PTMs into the nucleus where trans-splicing occurs.

Additional features can be added to the PTM molecule either after, or before, the nucleotide sequence encoding a translatable protein, such as polyadenylation signals to modify RNA expression/stability, or 5′ splice sequences to enhance splicing, additional binding regions, “safety”-self complementary regions, additional splice sites, or protective groups to modulate the stability of the molecule and prevent degradation. In addition, stop codons may be included in the PTM structure to prevent translation of unspliced PTMs. Further elements such as a 3′ hairpin structure, circularized RNA, nucleotide base modification, or synthetic analogs can be incorporated into PTMs to promote or facilitate nuclear localization and spliceosomal incorporation, and intra-cellular stability.

PTMs may also be generated that require a double-trans-splicing reaction for generation of a chimeric trans-spliced product. Such PTMs could, for example, be used to replace an internal exon or exons which could be used for expression of an apoA-1 variant protein. PTMs designed to promote two trans-splicing reactions are engineered as described above, however, they contain both 5′ donor sites and 3′ splice acceptor sites. In addition, the PTMs may comprise two or more binding domains and splice regions. The splice regions may be placed between the multiple binding domains and splice sites or alternatively between the multiple binding domains.

Optimal PTMs for wild type apoA-1 or other pre-mRNA targets, such as albumin pre-mRNA, may be selected by splicesome-mediated trans-splicing high capacity screens. Such screens include, but are not limited to, those described in patent application Ser. No. 10/693,192. Briefly, each new PTM library is clonally delivered to target cells by transfection of bacterial protoplasts or viral vectors encoding the PTMs. The 5′GFP-apoA-1, apoB, or albumin targets are transfected using Lipofectamine reagents and the cells analyzed for GFP expression by FACS. Total RNA samples may also be prepared and analyzed for trans-splicing by quantitative real time PCR (qRT-PCR) using target and PTM specific primers for the presence of correctly spliced repaired products and the level of repaired product. Each trans-splicing domain (TSD) and binding domain is engineered with several unique restriction sites, so that when a suitable sequence is identified (based on the level of GFP function and qRT-PCR data), part of or the complete TSD, can be readily subcloned into a PTM cassette to produce PTMs of the invention.

When specific PTMs are to be synthesized in vitro (synthetic PTMs), such PTMs can be modified at the base moiety, sugar moiety, or phosphate backbone, for example, to improve stability of the molecule, hybridization to the target mRNA, transport into the cell, etc. For example, modification of a PTM to reduce the overall charge can enhance the cellular uptake of the molecule. In addition modifications can be made to reduce susceptibility to nuclease or chemical degradation. The nucleic acid molecules may be synthesized in such a way as to be conjugated to another molecule such as a peptides (e.g., for targeting host cell receptors in vivo), or an agent facilitating transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life. Possible modifications include, but are not limited to, the addition of flanking sequences of ribonucleotides to the 5′ and/or 3′ ends of the molecule. In some circumstances where increased stability is desired, nucleic acids having modified internucleoside linkages such as 2′-O-methylation may be preferred. Nucleic acids containing modified internucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein).

The synthetic PTMs of the present invention are preferably modified in such a way as to increase their stability in the cells. Since RNA molecules are sensitive to cleavage by cellular ribonucleases, it may be preferable to use as the competitive inhibitor a chemically modified oligonucleotide (or combination of oligonucleotides) that mimics the action of the RNA binding sequence but is less sensitive to nuclease cleavage. In addition, the synthetic PTMs can be produced as nuclease resistant circular molecules with enhanced stability to prevent degradation by nucleases (Puttaraju et al., 1995, Nucleic Acids Symposium Series No. 33:49-51; Puttaraju et al., 1993, Nucleic Acid Research 21:4253-4258). Other modifications may also be required, for example to enhance binding, to enhance cellular uptake, to improve pharmacology or pharmacokinetics or to improve other pharmaceutically desirable characteristics.

Modifications, which may be made to the structure of the synthetic PTMs include but are not limited to backbone modifications such as use of:

(i) phosphorothioates (X or Y or W or Z=S or any combination of two or more with the remainder as O). e.g. Y=S (Stein, C. A., et al., 1988, Nucleic Acids Res., 16:3209-3221), X=S (Cosstick, R., et al., 1989, Tetrahedron Letters, 30:4693-4696), Y and Z=S (Brill, W. K.-D., et al., 1989, J. Amer. Chem. Soc., 111:2321-2322); (ii) methylphosphonates (e.g. Z=methyl (Miller, P. S., et al., 1980, J. Biol. Chem., 255:9659-9665); (iii) phosphoramidates (Z=N-(alkyl)₂ e.g. alkyl methyl, ethyl, butyl) (Z=morpholine or piperazine) (Agrawal, S., et al., 1988, Proc. Natl. Acad. Sci. USA 85:7079-7083) (X or W=NH) (Mag, M., et al., 1988, Nucleic Acids Res., 16:3525-3543); (iv) phosphotriesters (Z=O-alkyl e.g. methyl, ethyl, etc.) (Miller, P. S., et al., 1982, Biochemistry, 21:5468-5474); and (v) phosphorus-free linkages (e.g. carbamate, acetamidate, acetate) (Gait, M. J., et al., 1974, J. Chem. Soc. Perkin I, 1684-1686; Gait, M. J., et al., 1979, J. Chem. Soc. Perkin I, 1389-1394).

In addition, sugar modifications may be incorporated into the PTMs of the invention. Such modifications include the use of: (i) 2′-ribonucleosides (R═H); (ii) 2′-O-methylated nucleosides (R═OMe)) (Sproat, B. S., et al., 1989, Nucleic Acids Res., 17:3373-3386); and (iii) 2′-fluoro-2′-riboxynucleosides (R═F) (Krug, A., et al., 1989, Nucleosides and Nucleotides, 8:1473-1483).

Further, base modifications that may be made to the PTMs, including but not limited to use of: (i) pyrimidine derivatives substituted in the 5-position (e.g. methyl, bromo, fluoro etc) or replacing a carbonyl group by an amino group (Piccirilli, J. A., et al., 1990, Nature, 343:33-37); (ii) purine derivatives lacking specific nitrogen atoms (e.g. 7-deaza adenine, hypoxanthine) or functionalized in the 8-position (e.g. 8-azido adenine, 8-bromo adenine) (for a review see Jones, A. S., 1979, Int. J. Biolog. Macromolecules, 1:194-207).

In addition, the PTMs may be covalently linked to reactive functional groups, such as: (i) psoralens (Miller, P. S., et al., 1988, Nucleic Acids Res., Special Pub. No. 20, 113-114), phenanthrolines (Sun, J-S., et al., 1988, Biochemistry, 27:6039-6045), mustards (Vlassov, V. V., et al., 1988, Gene, 72:313-322) (irreversible cross-linking agents with or without the need for co-reagents); (ii) acridine (intercalating agents) (Helene, C., et al., 1985, Biochimie, 67:777-783); (iii) thiol derivatives (reversible disulphide formation with proteins) (Connolly, B. A., and Newman, P. C., 1989, Nucleic Acids Res., 17:4957-4974); (iv) aldehydes (Schiff's base formation); (v) azido, bromo groups (UV cross-linking); or (vi) ellipticines (photolytic cross-linking) (Perrouault, L., et al., 1990, Nature, 344:358-360).

In an embodiment of the invention, oligonucleotide mimetics in which the sugar and internucleoside linkage, i.e., the backbone of the nucleotide units, are replaced with novel groups can be used. For example, one such oligonucleotide mimetic which has been shown to bind with a higher affinity to DNA and RNA than natural oligonucleotides is referred to as a peptide nucleic acid (PNA) (for review see, Uhlmann, E. 1998, Biol. Chem. 379:1045-52). Thus, PNA may be incorporated into synthetic PTMs to increase their stability and/or binding affinity for the target pre-mRNA.

In another embodiment of the invention synthetic PTMs may covalently linked to lipophilic groups or other reagents capable of improving uptake by cells. For example, the PTM molecules may be covalently linked to: (i) cholesterol (Letsinger, R. L., et al., 1989, Proc. Natl. Acad. Sci. USA 86:6553-6556); (ii) polyamines (Lemaitre, M., et al., 1987, Proc. Natl. Acad. Sci. USA 84:648-652); other soluble polymers (e.g. polyethylene glycol) to improve the efficiently with which the PTMs are delivered to a cell. In addition, combinations of the above identified modifications may be utilized to increase the stability and delivery of PTMs into the target cell. The PTMs of the invention can be used in methods designed to produce a novel chimeric RNA in a target cell.

The methods of the present invention comprise delivering to the target cell a PTM which may be in any form used by one skilled in the art, for example, an RNA molecule, or a DNA vector which is transcribed into a RNA molecule, wherein said PTM binds to a pre-mRNA and mediates a trans-splicing reaction resulting in formation of a chimeric RNA comprising a portion of the PTM molecule spliced to a portion of the pre-mRNA. Furthermore, the invention also encompasses additional methods for modifying or converting mRNAs such as use of trans-splicing ribozymes and other means that are known to skilled practitioners in the field.

In a specific embodiment of the invention, the PTMs of the invention can be used in methods designed to produce a novel chimeric RNA in a target cell so as to result in expression of the apoA-1 Milano or other variant proteins. The methods of the present invention comprise delivering to a cell a PTM which may be in any form used by one skilled in the art, for example, an RNA molecule, or a DNA vector which is transcribed into a RNA molecule, wherein said PTM binds to a apoA-1 or apoB pre-mRNA and mediates a trans-splicing reaction resulting in formation of a chimeric RNA comprising the portion of the PTM molecule having the apo-1 Milano mutation spliced to a portion of the pre-mRNA.

In another specific embodiment of the invention, the PTMs of the invention can be used in methods designed to produce a novel chimeric RNA in a target cell so as to result in the substitution of albumin expression with expression of the wild type apoA-1, apoA-1 Milano or other variant proteins. The methods of the present invention comprise delivering to a cell a PTM which may be in any form used by one skilled in the art, for example, an RNA molecule, or a DNA vector which is transcribed into a RNA molecule, wherein said PTM binds to an albumin pre-mRNA and mediates a trans-splicing reaction resulting in formation of a chimeric RNA comprising the portion of the PTM molecule encoding wild type apoA-1, or apoA-1 Milano variant spliced to a portion of the pre-mRNA.

### Synthesis of the Trans-Splicing Molecules

The nucleic acid molecules of the invention can be RNA or DNA or derivatives or modified versions thereof, single-stranded or double-stranded. By nucleic acid is meant a PTM molecule or a nucleic acid molecule encoding a PTM molecule, whether composed of deoxyribonucleotides or ribonucleosides, and whether composed of phosphodiester linkages or modified linkages. The term nucleic acid also specifically includes nucleic acids composed of bases other than the five biologically occurring bases (adenine, guanine, thymine, cytosine and uracil). In addition, the PTMs of the invention may comprise, DNA/RNA, RNA/protein or DNA/RNA/protein chimeric molecules that are designed to enhance the stability of the PTMs.

The PTMs of the invention can be prepared by any method known in the art for the synthesis of nucleic acid molecules. For example, the nucleic acids may be chemically synthesized using commercially available reagents and synthesizers by methods that are well known in the art (see, e.g., Gait, 1985, Oligonucleotide Synthesis: A Practical Approach, IRL Press, Oxford, England).

Alternatively, synthetic PTMs can be generated by in vitro transcription of DNA sequences encoding the PTM of interest. Such DNA sequences can be incorporated into a wide variety of vectors downstream from suitable RNA polymerase promoters such as the T7, SP6, or T3 polymerase promoters. Consensus RNA polymerase promoter sequences include the following:

| | |
|---|---|
| T7: | TAATACGACTCACTATAGGGAGA |
| SP6: | ATTTAGGTGACACTATAGAAGNG |
| T3: | AATTAACCCTCACTAAAGGGAGA. |

The base in bold is the first base incorporated into RNA during transcription. The underline indicates the minimum sequence required for efficient transcription.

RNAs may be produced in high yield via in vitro transcription using plasmids such as SPS65 and Bluescript (Promega Corporation, Madison, Wis.). In addition, RNA amplification methods such as Q-β amplification can be utilized to produce the PTM of interest.

The PTMs may be purified by any suitable means, as are well known in the art. For example, the PTMs can be purified by gel filtration, affinity or antibody interactions, reverse phase chromatography or gel electrophoresis. Of course, the skilled artisan will recognize that the method of purification will depend in part on the size, charge and shape of the nucleic acid to be purified.

The PTM's of the invention, whether synthesized chemically, in vitro, or in vivo, can be synthesized in the presence of modified or substituted nucleotides to increase stability, uptake or binding of the PTM to a target pre-mRNA. In addition, following synthesis of the PTM, the PTMs may be modified with peptides, chemical agents, antibodies, or nucleic acid molecules, for example, to enhance the physical properties of the PTM molecules. Such modifications are well known to those of skill in the art.

In instances where a nucleic acid molecule encoding a PTM is utilized, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The DNA encoding the PTM of interest may be recombinantly engineered into a variety of host vector systems that also provide for replication of the DNA in large scale and contain the necessary elements for directing the transcription of the PTM. The use of such a construct to transfect target cells in the patient will result in the transcription of sufficient amounts of PTMs that will form complementary base pairs with the endogenously expressed pre-mRNA targets, such as for example, apoA-1 or apoB pre-mRNA target, and thereby facilitate a trans-splicing reaction between the complexed nucleic acid molecules. For example, a vector can be introduced in vivo such that is taken up by a cell and directs the transcription of the PTM molecule. Such a vector can remain episomal or become chromosomally integrated, as long as it can be transcribed to produce the desired RNA, i.e., PTM. Such vectors can be constructed by recombinant DNA technology methods standard in the art.

Vectors encoding the PTM of interest can be plasmid, viral, or others known in the art, used for replication and expression in mammalian cells. Expression of the sequence encoding the PTM can be regulated by any promoter/enhancer sequences known in the art to act in mammalian, preferably human cells. Such promoters/enhancers can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42), the viral CMV promoter, the human chorionic gonadotropin-β promoter (Hollenberg et al., 1994, Mol. Cell. Endocrinology 106:111-119), etc.

In a specific embodiment of the invention, liver specific promoter/enhancer sequences may be used to promote the synthesis of PTMs in liver cells for expression of the apoA-1 Milano variant protein. Such promoters include, for example, the albumin, transthyretin, CMV enhancers/chicken beta-actin promoter, ApoE enhancer alpha1-antitrypsin promoter and endogenous apoA-1 or apo-B promoter elements. In addition, the liver-specific microglobulin promoter cassette optimized for apoA-1 or apo-B gene expression may be used, as well as, post-transcriptional elements such as the wood chuck post-transcriptional regulatory element (WPRE).

Any type of plasmid, cosmid, YAC or viral vector can be used to prepare the recombinant DNA construct which can be introduced directly into the tissue site. Alternatively, viral vectors can be used which selectively infect the desired target cell. Vectors for use in the practice of the invention include any eukaryotic expression vectors, including but not limited to viral expression vectors such as those derived from the class of retroviruses, adenoviruses or adeno-associated viruses.

A number of selection systems can also be used, including but not limited to selection for expression of the herpes simplex virus thymidine kinase, hypoxanthine-guanine phosphoribosyltransterase and adenine phosphoribosyl transferase protein in tk-, hgprt- or aprt-deficient cells, respectively. Also, anti-metabolic resistance can be used as the basis of selection for dihydrofolate transferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin. In a preferred embodiment of the invention, the cell culture is transformed at a low ratio of vector to cell such that there will be only a single vector, or a limited number of vectors, present in any one cell.

### Uses and Administration of Trans-Splicing Molecules

### Use of PTM Molecules for Expression of ApoA-1 Milano Variants

The compositions and methods of the present invention are designed to substitute apoA-1, or apoB expression, or other pre-mRNA targets, such as albumin, with wild-type apoA-1, apoA-1 Milano or other apoA-1 variant expression. Specifically, targeted trans-splicing, including double-trans-splicing reactions, 3′ exon replacement and/or 5′ exon replacement can be used to substitute apoA-1, apoB, or albumin sequences with either wild type apoA-1 or apoA-1 Milano sequences resulting in expression of ApoA-1 wild type or Milano variant.

Various delivery systems are known and can be used to transfer the compositions of the invention into cells, e.g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral, adenoviral, adeno-associated viral or other vector, injection of DNA, electroporation, calcium phosphate mediated transfection, etc.

The compositions and methods can be used to provide a gene encoding a wild-type apoA-1, apoA-1 Milano, apoB/apoA-1 wild type or Milano, alb/apoA-1 wild type or milano fusion protein to cells of an individual where expression of said gene products reduces plaque formation.

Specifically, the compositions and methods can be used to provide sequences encoding a wild type apoA-1, an apoA-1 Milano variant molecule, or apoB/apoA-1 or alb/apoA-1 fusion protein to cells of an individual to reduce the plaque formation normally associated with vascular disorders leading to heart attacks and stroke.

In a preferred embodiment, nucleic acids comprising a sequence encoding a PTM are administered to promote PTM function, by way of gene delivery and expression into a host cell. In this embodiment of the invention, the nucleic acid mediates an effect by promoting PTM production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Delivery of the PTM into a host cell may be either direct, in which case the host is directly exposed to the PTM or PTM encoding nucleic acid molecule, or indirect, in which case, host cells are first transformed with the PTM or PTM encoding nucleic acid molecule in vitro, then transplanted into the host. These two approaches are known, respectively, as in vivo or ex vivo gene delivery.

In a specific embodiment, the nucleic acid is directly administered in vivo, where it is expressed to produce the PTM. This can be accomplished by any of numerous methods known in the art, e.g., by constructing it as part of an appropriate nucleic acid expression vector and administering it so that it becomes intracellular, e.g. by infection using a defective or attenuated retroviral or other viral vector (see U.S. Pat. No. 4,980,286), or by direct injection of naked DNA, or by use of microparticle bombardment (e.g., a gene gun; Biolistic, Dupont, Bio-Rad), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (see, Kozarsky and Wilson, 1993, Current Opinion in Genetics and Development 3:499-503 for a review of adenovirus-based gene delivery).

In a preferred embodiment of the invention an adeno-associated viral vector may be used to deliver nucleic acid molecules capable of encoding the PTM. The vector is designed so that, depending on the level of expression desired, the promoter and/or enhancer element of choice may be inserted into the vector.

Another approach to gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host's cell.

In a specific embodiment of the invention, hepatic stem cells, oval cells, or hepatocytes may be removed from a subject and transfected with a nucleic acid molecule capable of encoding a PTM designed to produce, upon trans-splicing, a wild-type apoA-1, an apoA-1 Milano or other apoA-1 variant protein and/or apoB/apoA-1 or alb/apoA-1 fusion protein. Cells may be further selected, using routine methods known to those of skill in the art, for integration of the nucleic acid molecule into the genome thereby providing a stable cell line expressing the PTM of interest. Such cells are then transplanted into the subject thereby providing a source of wild type apoA-1, or apoA-1 Milano variant protein.

The present invention also provides for pharmaceutical compositions comprising an effective amount of a PTM or a nucleic acid encoding a PTM, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin.

In specific embodiments, pharmaceutical compositions are administered: to subjects with diseases or disorders involving accumulation of plaque in the vascular system, for example, in hosts where aberrant levels of apoA-1 and apoB protein are expressed. The activity of the protein encoded for by the chimeric mRNA resulting from the PTM mediated trans-splicing reaction can be readily detected, e.g., by obtaining a host tissue sample (e.g., from biopsy tissue, or a blood sample) and assaying in vitro for mRNA or protein levels or activity of the expressed chimeric mRNA.

In specific embodiments, pharmaceutical compositions are administered in diseases or disorders involving the accumulation of plaque in the vascular system, for example, in hosts where apoA-1 and/or apoB are aberrantly expressed. Such disorders include but are not limited to vascular disorders that frequently lead to heart attacks or strokes.

Many methods standard in the art can be thus employed, including but not limited to immunoassays to detect and/or visualize the protein, i.e., wild type apoA-1, apoA-1 Milano or apoB/apoA-1 Milano fusion protein, encoded for by the chimeric mRNA (e.g., Western blot, immunoprecipitation followed by sodium dodecyl sulfate polyacrylamide gel electrophoresis, immunocytochemistry, etc.) and/or hybridization assays to detect formation of chimeric mRNA expression by detecting and/or visualizing the presence of chimeric mRNA (e.g., Northern assays, dot blots, in situ hybridization, and Reverse-Transcription PCR, etc.), etc.

In a specific embodiment, it may be desirable to administer the pharmaceutical compositions of the invention locally to the area in need of treatment, i.e., liver tissue. This may be achieved by, for example, and not by way of limitation, local infusion during surgery, topical application, e.g., in conjunction with a wound dressing after surgery, by injection, by means of a catheter or stent, by means of a suppository, or by means of an implant, said implant being of a porous, non-porous, or gelatinous material, including membranes, such as sialastic membranes, or fibers. Other control release drug delivery systems, such as nanoparticles, matrices such as controlled-release polymers, hydrogels.

The PTM will be administered in amounts which are effective to produce the desired effect in the targeted cell. Effective dosages of the PTMs can be determined through procedures well known to those in the art which address such parameters as biological half-life, bioavailability and toxicity. The amount of the composition of the invention which will be effective will depend on the severity of the vascular disorder being treated, and can be determined by standard clinical techniques. Such techniques include analysis of blood samples to determine the level of apoA-1 or ApoB/apoA-1 or alb/apoA-1 fusion protein expression. In addition, in vitro assays may optionally be employed to help identify optimal dosage ranges.

The present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLE: EXPRESSION OF HUMAN APOLIPOPROTEIN (APO A-1)

### Albumin-HUMAN Apo A-1 Fusion Proteins

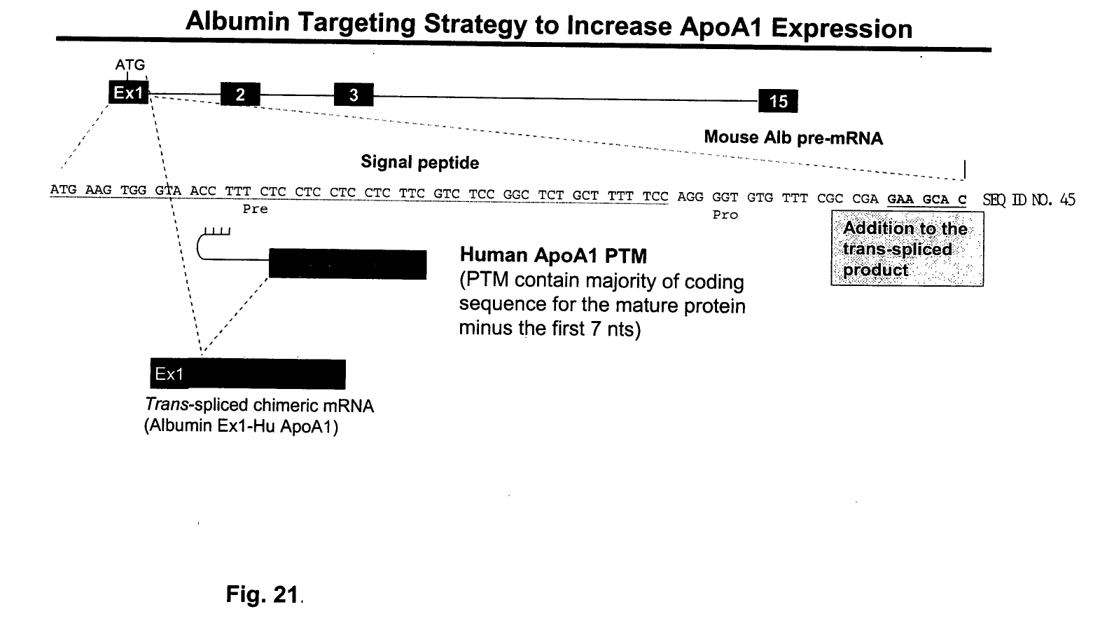

The present study was undertaken to evaluate albumin targeting strategy (FIG. 21) for the production of human Apo A-1 protein, major component of high density lipoprotein (HDL) or other variants and subsequently increase HDL concentration as a treatment for individuals having or at risk for cardio vascular disease (CHD). The rationale for selecting albumin as a target is because of its elevated expression in liver. High albumin pre-mRNA concentration results in abundant targets for trans-splicing. The concept involves targeted trans-splicing of wild type human Apo A-1 or Apo A-1 analogues into albumin pre-mRNA target; and the goal is to increase Apo A-1 expression. This study evaluates the effect of albumin sequence human Apo A-1 protein expression, secretion and function.

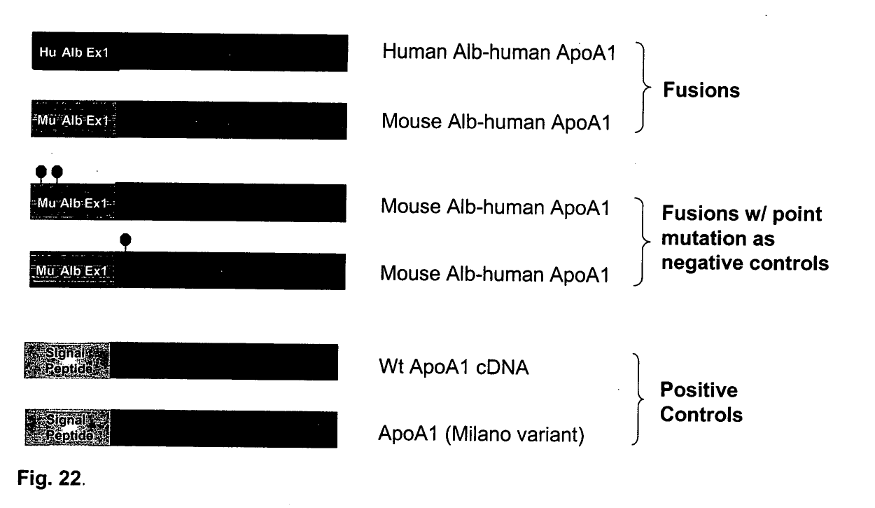

The fusion (albumin-Apo A-1) function in vivo was evaluated. Human and mouse versions of the albumin-human Apo A-1 cDNA controls (FIG. 22) were constructed to mimic the final trans-spliced product for expression, processing and function in 293 and hepatoma cells (HepG2). The fusion cDNA constructs were constructed using long complementary oligonucleotides and PCR products consisting of albumin exon 1 and human Apo A-1 exon 3 and 4. Briefly, the coding sequence of mouse and human albumin exon 1 were assembled using the following long oligos:

mouse Alb forward primer:

ATGAAGTGGGTAACCTTTCTCCTCCTCCTCTTCGTCTCCGGCTCTGCTTTTTCCAGGG
GTGTGTTTCGCCGAGAAGCACCC,

reverse primer:

GGGTGCTTCTCGGCGAAACACACCCCTGGAAAAAGCAGAGCCGGAGACGAAGAGG
AGGAGGAGAAAGGTTACCCACTTCATG,

and human Alb forward primer:

ATGAAGTGGGTAACCTTTATTTCCCTTCTTTTTCTCTTTAGCTCGGCTTATTCC
AGGGGTGTGTTTCGTCGAGATGCACCC,

reverse primer:

GGGTGCATCTCGACGAAACACACCCCTGGAATAAGCCGAGCTAAAGAGAAAAAGA
AGGGAAATAAAGGTTACCCACTTCATG.

The underlined nucleotides indicate the end of albumin exon 1 sequence and 2 “C”s at the 3′ end of the forward primers overlap to human Apo A-1.

Human Apo A-1 coding sequence was PCR amplified using a cDNA clone (ATCC: clone # MGC-1249) and primers: Apo23 (5′-CCCCAGAGCCCCTGGGATCGAGTG) and Apo5 (5′-CTAG AAGCTT CCCACTTTGGAAACGTTTAT TCTGAGCACC GG). The PCR product was blunted at the 5′ end and then digested with Hind III (indicated in bold) restriction enzyme. The resulting product was first ligated with mouse or human albumin exon 1 and then cloned into pcDNA3.1 expression vector (Invitrogen). Expression plasmids containing the entire coding sequence of human Apo A-1 including the signal peptide into pcDNA3.1 to generate wild type human Apo A-1, and the Milano variant which contains an Arg to Cys substitution at position 173 (R173C) expression plasmids were also constructed as positive controls. The final constructs were verified by sequencing.

### Production, Expression and Secretion of Albumin Apo A-1 Fusion Proteins in 293 Cells

The effect of albumin exon 1 sequence on expression and processing of human Apo A-1 protein was evaluated by transfecting human and mouse fusion cDNA plasmids along with a negative (deletion mutant) and a positive control cDNAs (wt Apo A-1) into 293 cells. After transfection, cells were rinsed 2× with serum free DMEM and incubated with serum free advanced DMEM media (Invitrogen). After 48 hrs post-transfection, media was collected, concentrated, analyzed for the expression of human Apo A-1 protein.

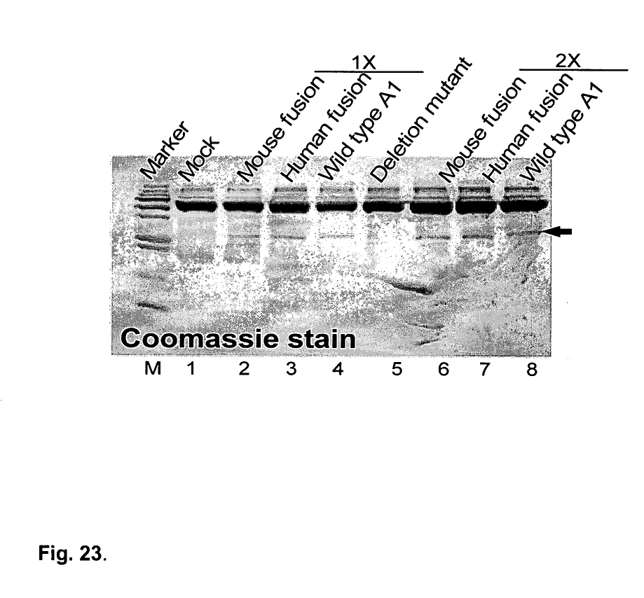

Coomassie Blue staining of the gel revealed that both the mouse and the human fusion cDNAs produced the predicted ˜28 kDa protein band which co-migrated with that of wt Apo A-1 demonstrating good expression, processing and secretion in 293 cells (FIG. 23. lanes 2-3,6-7). In addition, these data also showed that the level of expression was similar to that of wt Apo A-1 (FIG. 23. lane 4, 8) indicating no adverse effects of albumin sequence on human Apo A-1 expression and processing. On the other hand, no such band was detected in mock and in cells that received mouse fusion cDNA with 2 nucleotide deletion in the signal peptide (FIG. 23. lane 1 and 5).

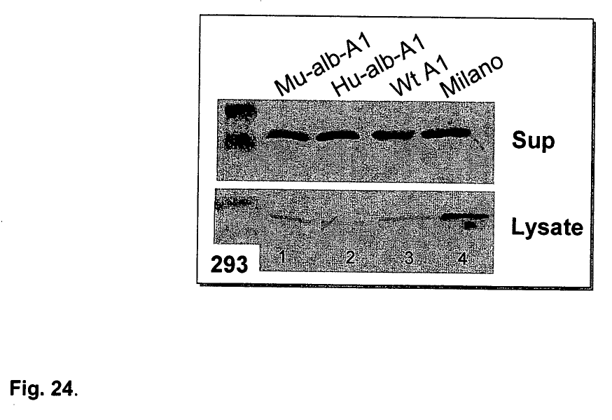

The identity of the band that was observed in SDS gel as human Apo A-1 was confirmed by Western analysis using a monoclonal human Apo A-1 antibody (Biodesign, Cat. # H45625). About ˜5-10 μg total protein from the supernatant or the total cell lysate from cells transfected with fusion cDNA constructs, wt Apo A-1 and Milano variant was analyzed on a 12% SDS gel and transferred onto nylon membrane and incubated with human anti-Apo A-1 antibody. Western results confirmed the production of human Apo A-1 protein with an apparent molecular mass of 28 kDa predicted for the mature protein. Western data also indicated the presence of >90% of the mature human Apo A-1 protein from the fusions or wt Apo A-1 in the supernatant compared to cell lysate demonstrating normal processing and secretion in 293 cells (FIG. 24; compare lanes 1 & 2 with 3). Similar results were also observed with hepatoma (HepG2) cells transfected with fusion cDNA constructs.

### Albumin Apo A-1 Fusion Protein is Functionally Active

The effect of albumin sequence on human Apo A-1 function was evaluated by measuring ATP-binding cassette transporter protein (ABC 1) mediated transfer of cellular cholesterol into Apo A-1 acceptor. The release of radio-labeled cellular cholesterol to lipid free human Apo A-1 was quantified and the efflux values obtained with fusion proteins was compared with those from wt Apo A-1 and negative control samples. Control HeLa and HeLa cells stably transfected with ABC1 plasmid were grown to near confluency. Cells were then loaded with 1 μCi/ml ³H cholesterol. After equilibrating for 24 hrs, cells were washed 3× with serum free media and incubated with a serial dilution of the media containing the fusion proteins (supernatant from 293 cells transfected w/fusion cDNA constructs, normalized for Apo A-1 protein concentration) or with 10 μg/wild type Apo A-1 protein as positive control. Cells were allowed to efflux for 18 hrs. After the efflux period, media was collected and an aliquot of the medium was then counted by liquid scintillation counting. The remaining counts in the cell fraction were determined after an over night extraction with isopropanol. The percent efflux was calculated by dividing the counts in the efflux media by the sum of the counts in the media plus the cell fraction. DMEM/BSA media was used as a blank and was subtracted from the radioactive counts obtained in the presence of an acceptor in the efflux media.

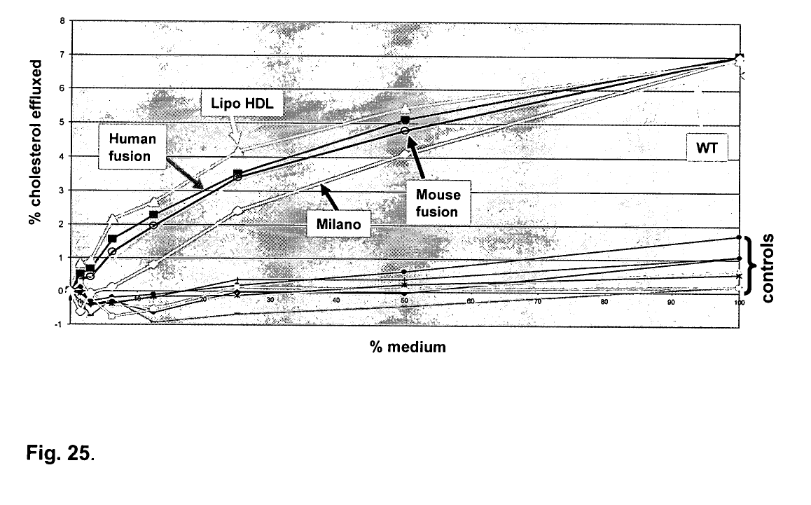

The amount of ABC1 mediated efflux observed with fusion proteins (mouse and human fusion proteins) was similar to that of wt Apo A-1 (FIG. 25). The efflux data also demonstrated that the absolute efflux activity observed with the fusion proteins were comparable or slightly better than the wt Apo A-1 protein across the concentration range tested indicating the absence of any major adverse effects due to albumin sequence in the final trans-spliced product on Apo A-1 function. These results provide strong evidence about the effectiveness of the compositions of the present invention for the production of functional biologically active proteins in vivo.

### EXAMPLE: HIGH CAPACITY SCREENS FOR ISOLATION OF OPTIMAL BINDING DOMAINS FOR ALBUMIN TARGETS

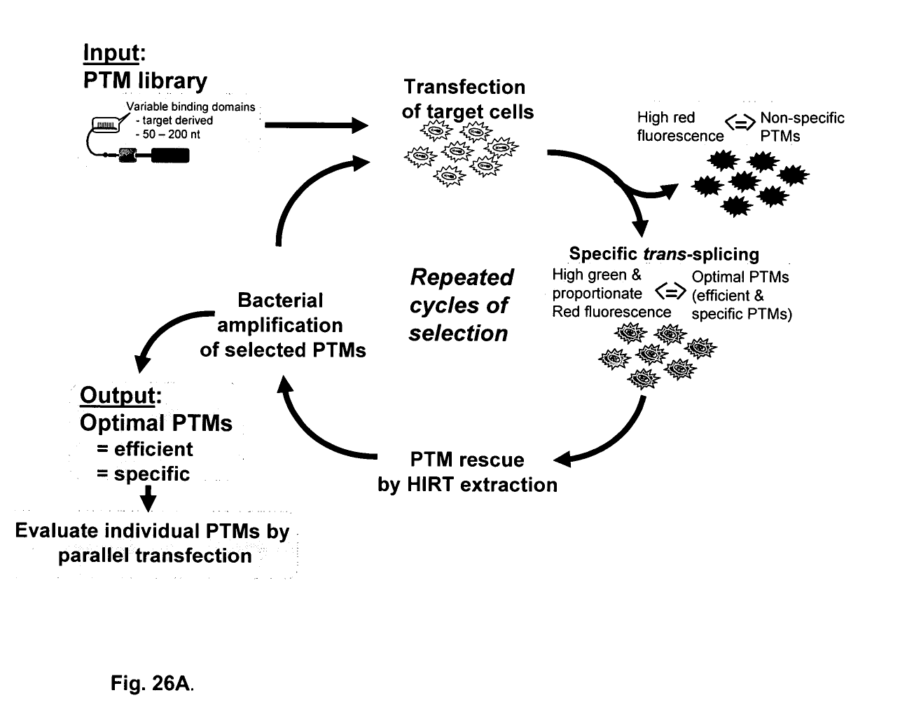

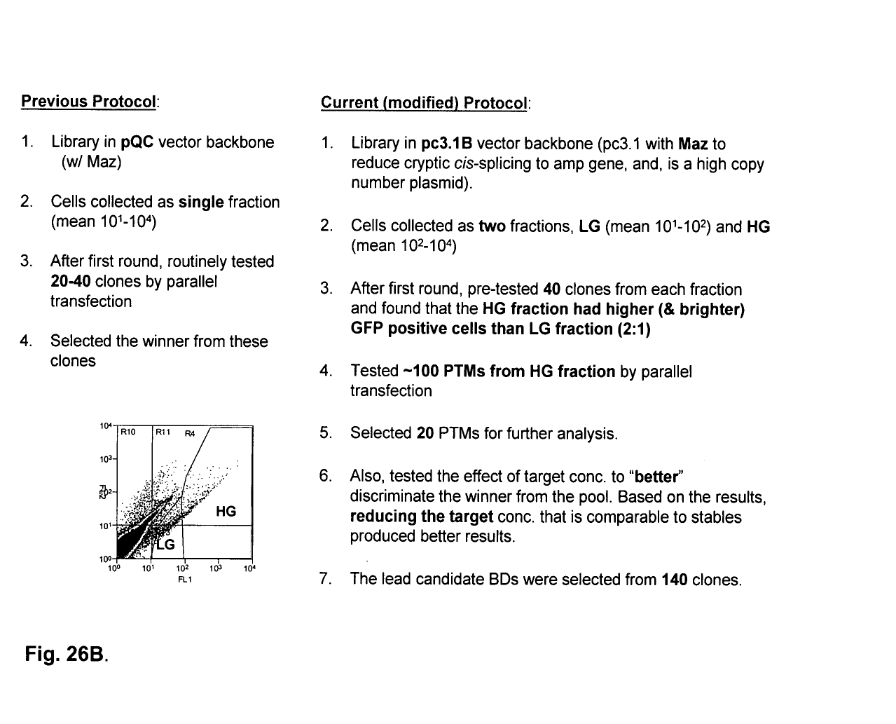

A high capacity screen (HCS) to identify optimal binding domains for mouse albumin pre-mRNA target was performed as described before (U.S. patent application Ser. No. 10/693,192, filed Oct. 24, 2003) (FIG. 26A) with various modifications (FIG. 26B).

### High Capacity Screen Pre-mRNA Target

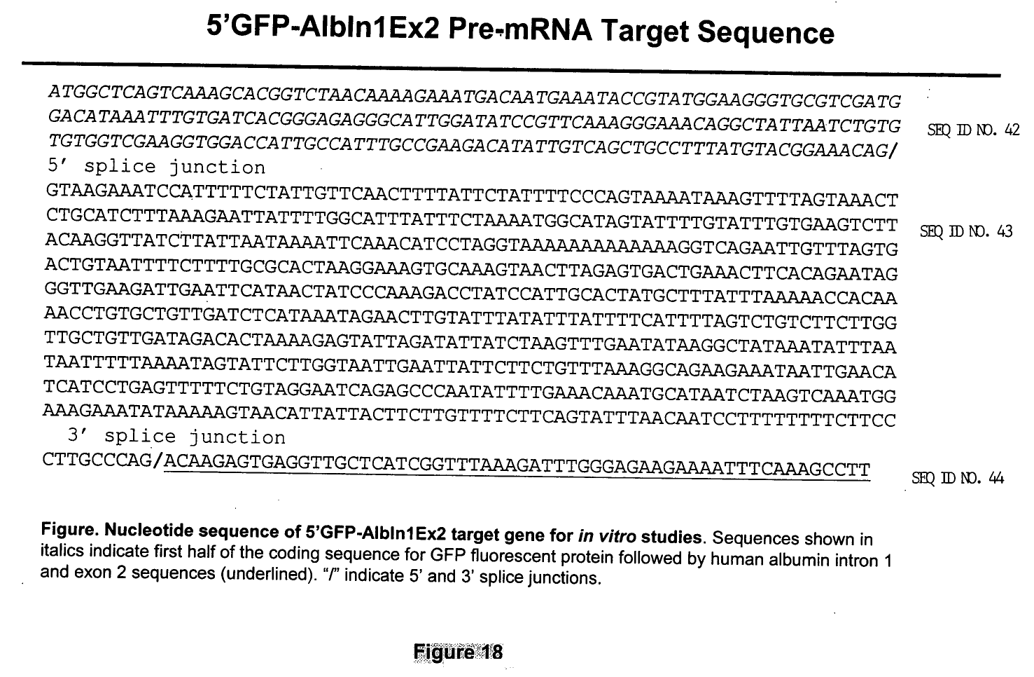

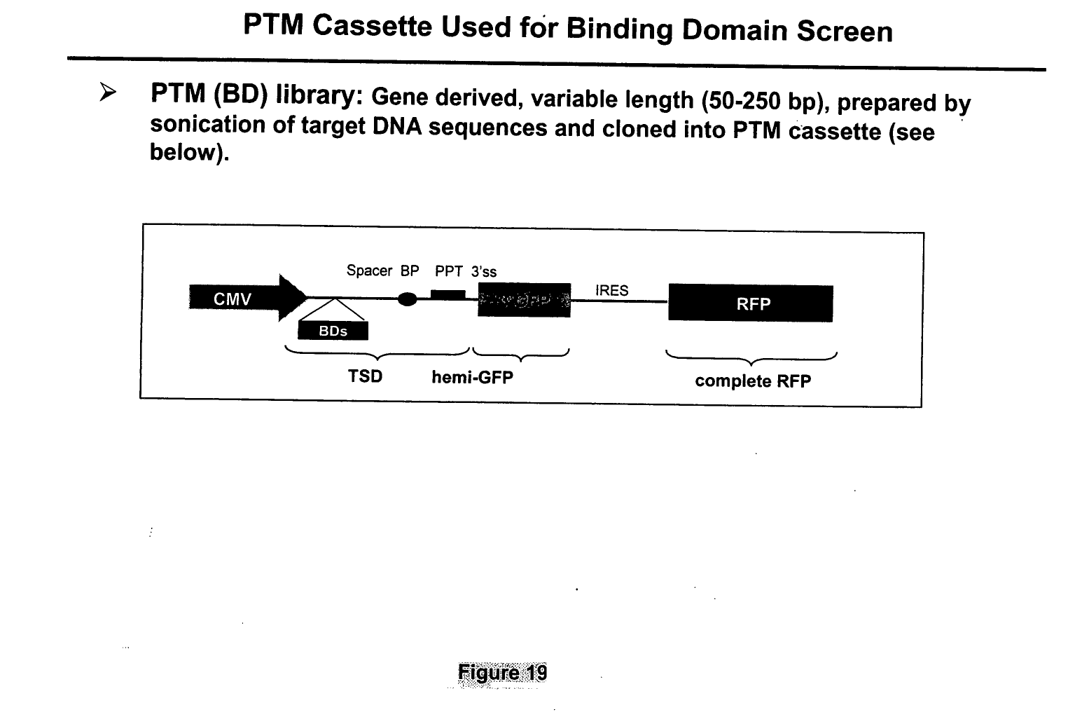

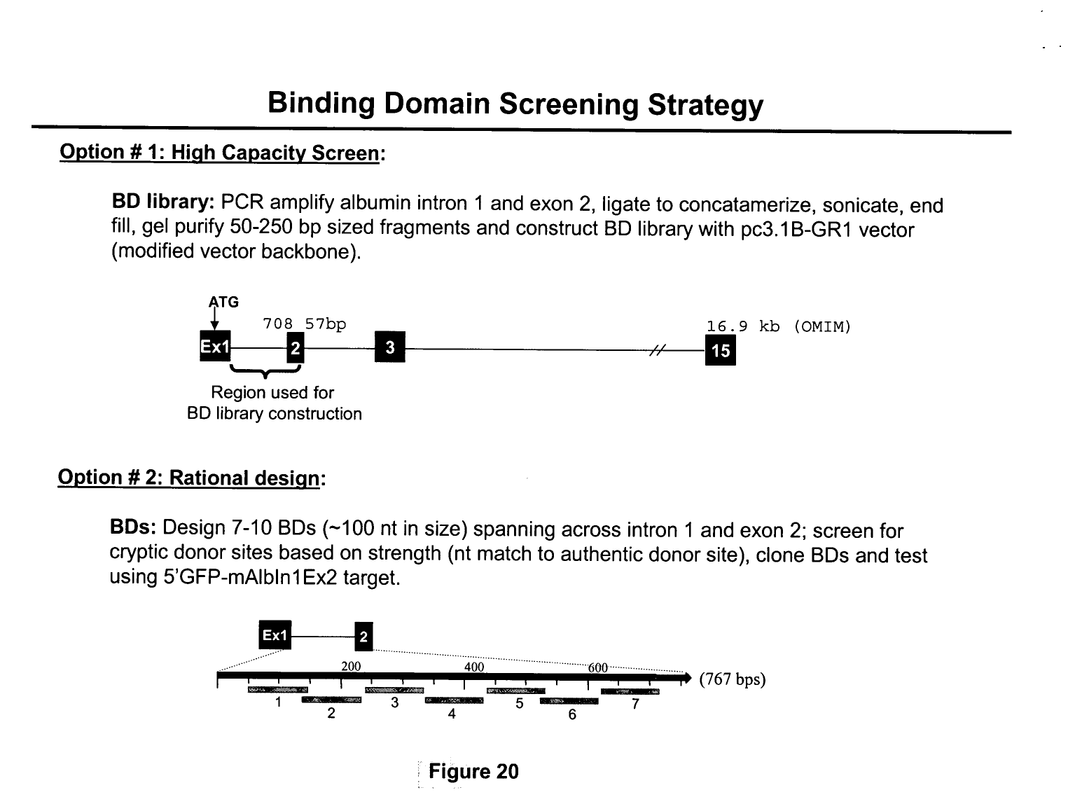

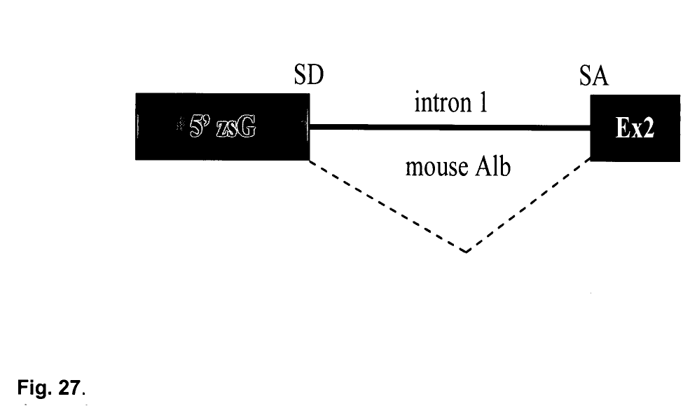

Mouse albumin intron 1 and exon 2 comprising of nucleotides 114 through 877 total of 763 bp (Ref. seq. NC_{—}000071) (FIG. 18) was PCR amplified using the genomic DNA and primers mAlb15 (5′-CTAG GGATCC GTTTTATGTTTTTTCATCTCTG) and mAlb8 (5′-CTAG GCGGCCGC_AGGCCTTTGAAATGTTGTTCTCC). The PCR product was then digested with Bam HI and Not I (indicated in bold) and cloned into an existing HCS target plasmid to generate pc5′zsG-mIn1-Ex2 plasmid (FIG. 27). Stable cells expressing the 5′ half of the coding sequence for the green fluorescent protein (GFP) (zsGreen from Clontech) coupled to intron 1 and exon 2 of mouse albumin gene was established in 293 cells by transfecting the target plasmid followed by hygromycin selection. After 2 weeks of selection, hygromycin resistant clones were pooled, characterized by RT-PCR and used for HCS.

### Mouse Albumin PTM Binding Domain Library

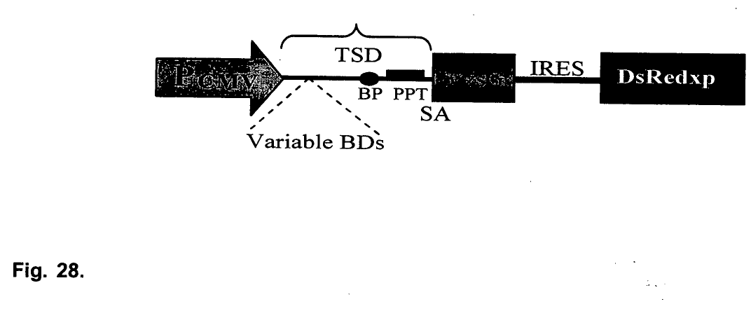

The mouse albumin sequence comprising intron 1 and exon 2 was PCR amplified using genomic DNA and primers as described above, digested with Bam HI and Not I and ligated to generate a large concatemerized fragment (˜10 kb). This step was introduced to increase BD complexity. The concatemerized DNA was then fragmented into small pieces by sonication and fractionated on a 3% agarose gel. Fragment size ranging from 50-250 nucleotides were gel purified, ends were repaired using Klenow enzyme and cloned into PTM cassette described before (U.S. patent application Ser. No. 10/693,192, filed Oct. 24, 2003) (FIG. 28).

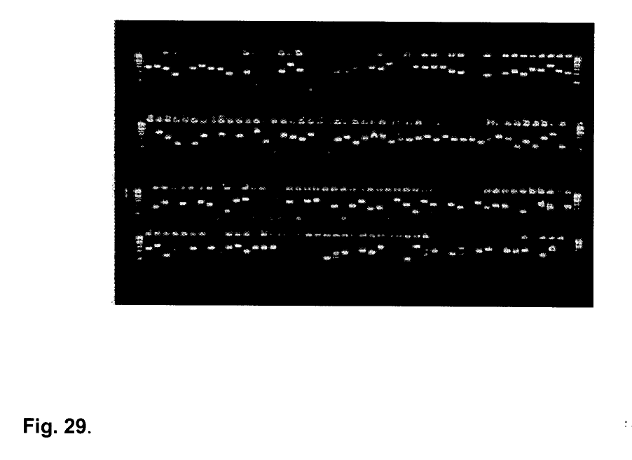

PCR analysis of the library colonies showed >87% recombination efficiency and produced a complex library with >106 independent clones with BDs varying in size from 50-250 nts (FIG. 29). The primary library was amplified in bacteria and used for screening the optimal BDs by HCS.

### PTM Selection Strategy

Following the FACS-based PTM selection strategy described before (U.S. patent application Ser. No. 10/693,192, filed Oct. 24, 2003), a mAlb binding domain (BD) library using the assay cells expressing the 5′zsG-mIn1-Ex2 pre-mRNA target was tested. Several of the existing steps were modified and several new steps were added as outlined in FIG. 26B.

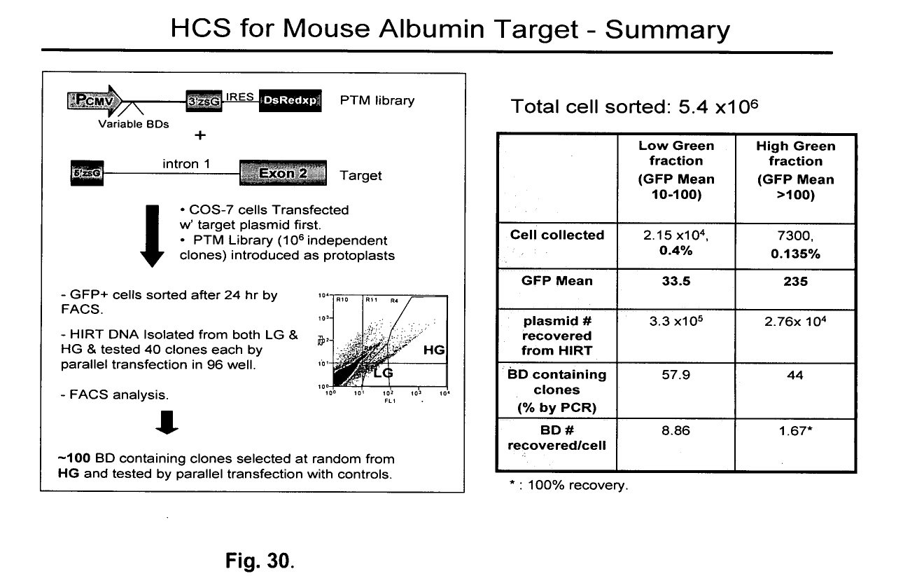

Briefly, on day 1, COS-7 cells were plated and transfected with 5′zsG-mIn1-Ex2 target plasmid using Lipo200 reagent. On day 2, ˜10⁶ independent PTM clones were delivered to assay cells expressing 5′zsG-mIn1-Ex2 pre-mRNA as protoplasts. As illustrated in the FIG. 30, cells were sorted after 24 hr by FACS, and cells expressing high GFP and proportionate RFP were collected in 2 fractions i.e., high green (HG) and low green (LG) fractions, instead of a single fraction as previously described. PTMs from the collected cells were rescued by HIRT DNA extraction followed by EcoR V digestion to reduce target plasmid contamination in the final HIRT DNA preparation. About 40 binding domain containing PTMs from LG and HG fractions were initially tested by parallel transfection. Trans-splicing efficiency of these PTMs was assessed by FACS analysis.

As predicted, the percent GFP positive (GFP⁺) cells and the mean GFP fluorescence was higher in PTMs from HG fraction compared to LG fraction with a 2:1 ratio (FIG. 30).

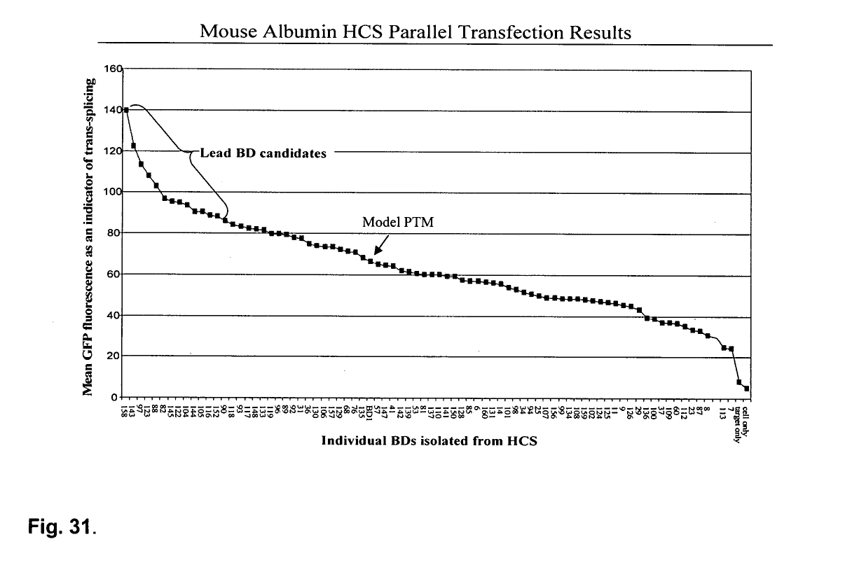

A hundred more BD containing clones from HG fraction was isolated and tested by parallel transfection and the results are summarized in FIG. 31. GFP mean fluorescence was used as an indicator for assessing trans-splicing efficiency of the individual PTMs. Based on the GFP mean fluorescence, the trans-splicing efficiency of the majority of the PTMs selected from the HCS were either similar or slightly higher than the rationally designed model PTM (FIG. 31). However, several PTMs with considerably higher (1.5 to 2-fold) trans-splicing compared with the model PTM were present. In the current screen, a ratio of 1:20 of superior PTMs vs. the rest was obtained.

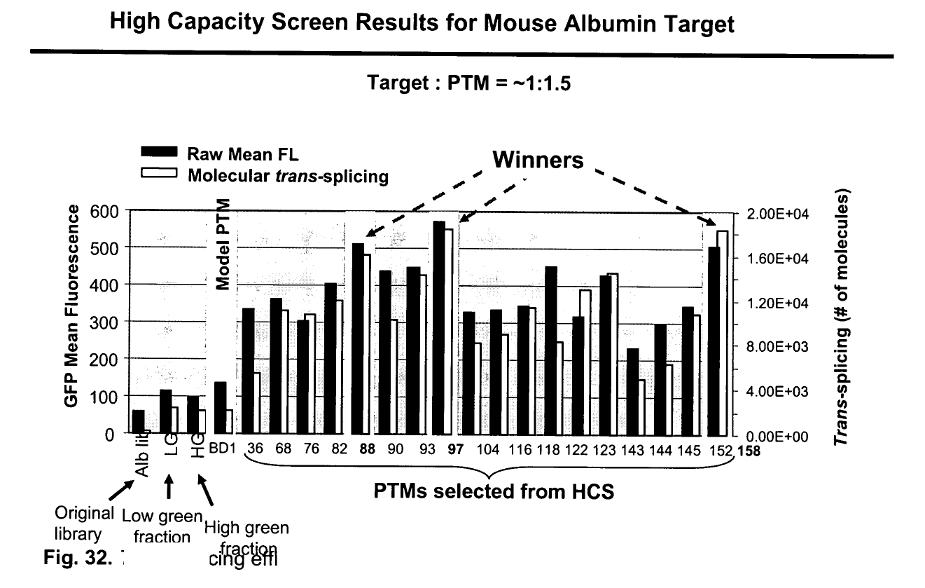

From this step, the top 20 PTMs were selected for further characterization by parallel transfection followed by molecular analysis using reverse transcription (RT) real time quantitative PCR (RT-qPCR) for specific trans-splicing and the results are summarized in FIG. 32. Total RNA was isolated and trans-splicing efficiency was measured by RT-qPCR. Target and PTM specific primers were used for measuring specific trans-splicing, and total splicing was measured using primers specific for the 5′zsG exon as previously described. Based on the qPCR or GFP mean fluorescence values up to ˜5-10 fold enrichment (after normalization) for trans-splicing efficiency was detected with PTMs selected from the HCS compared to a rationally designed model PTM (FIG. 32). Similar results, i.e. enhancement in trans-splicing efficiency, was observed with the enriched library (LG and HG samples) compared with the starting library, which is consistent with previous screen.

The effect of BD orientation and sequence position on trans-splicing efficiency and specificity was also analyzed. The sequence of random clones from the starting PTM library were compared with the enriched library i.e., PTMs selected after one round of enrichment.

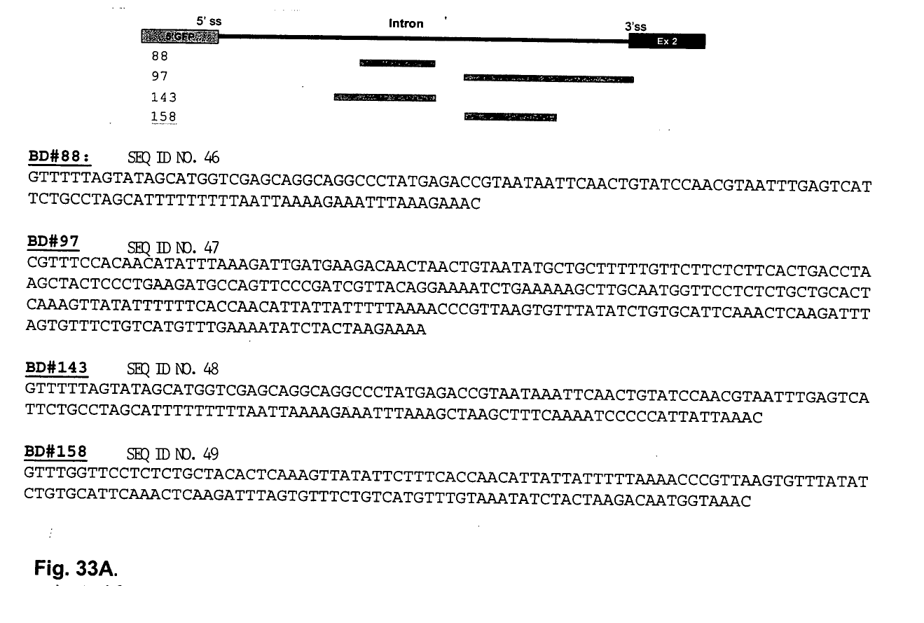

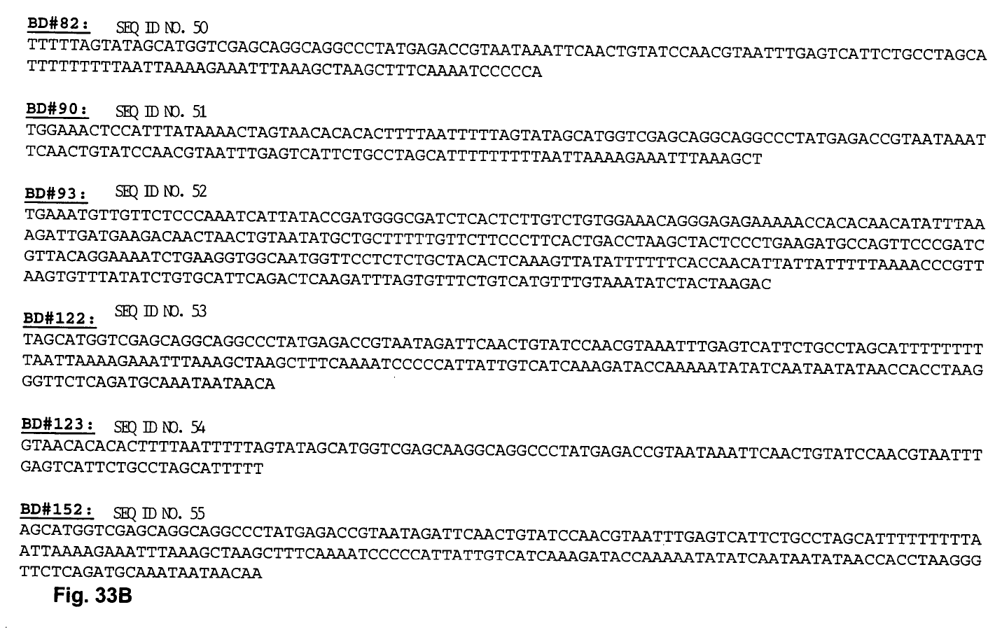

Sequence analysis of the PTMs from the starting library revealed that 51% of the BDs were in correct (antisense) orientation compared to 49% incorrect orientation. The BD size varied from 40 nt and up to 336 nt and also showed good distribution indicating the complexity of the mAlb BD library. In contrast, sequence analysis of the PTMs selected from the enriched library, as expected, showed an increase in correct orientation BDs (88%) and the mean BD length was significantly higher than the starting library, which is consistent with previous work demonstrating that longer BDs are more efficient (Puttaraju et al., 2001). Based on molecular and GFP mean fluorescence values, lead PTMs # 88, 97, 143 and 158 were selected for functional studies. Sequence and relative positions of these lead PTMs are shown in FIG. 33. In addition to the lead PTMs mentioned above, several PTMs with significantly higher trans-splicing were selected and compared with model PTMs. Examples include: 82, 90, 93, 122, 123 and 152 (FIG. 33A).

### EXAMPLE: TRANS-SPLICING OF HUMAN APOLIPOPROTEIN APO A-1 IN CELLS

### Human Apolipoprotein (Apo A-1) PTM

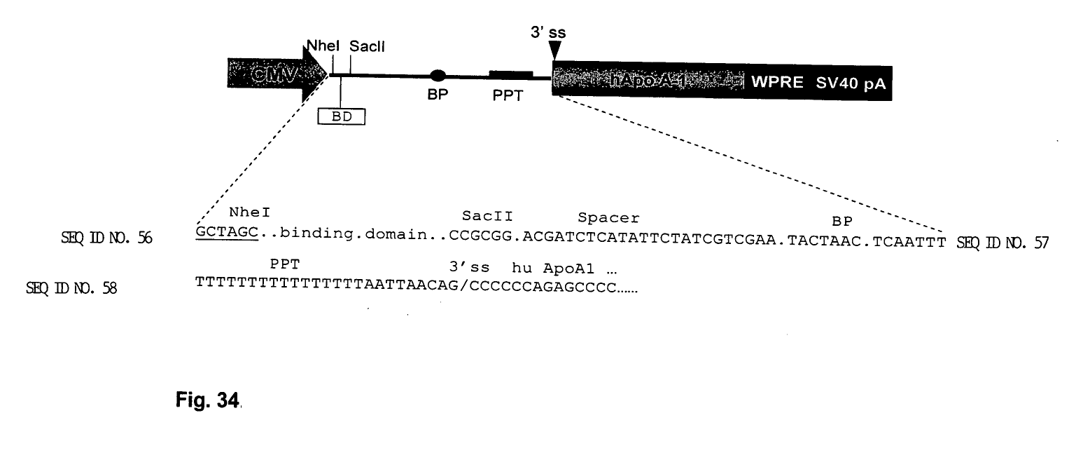

Detailed structure of a human Apolipoprotein A1 (Apo A-1) PTM used in this example to show proof of principle is shown in FIG. 34. The PTM cassette consists of a trans-splicing domain (TSD) that include unique restriction sites, NheI and SacII, for cloning the lead binding domains (BDs), a 24 nucleotide spacer region, a strong 3′ splice site including the consensus yeast branch point (BP), an extended polypyrimidine tract (19 nucleotides long), a splice acceptor site (CAG dinucleotide) followed by the majority of the coding sequence for wild type human Apo A-1 mRNA from nt 118 through nt 842 (Ref seq. NM_{—}000039 and as shown in FIG. 3A). The PTM cassette also contains the SV40 polyadenylation site and woodchuck hepatitis post-transcriptional regulatory element (WPRE) to enhance the stability of trans-spliced message. The entire cassette is cloned into pcDNA3.1 vector backbone, which contains cytomegalovirus promoter (Invitrogen). In addition, the vector backbone was further modified to include Maz4 (transcriptional pause site) sequence to reduce cryptic cis-splicing between vector ampicillin gene and PTM 3′ splice site. PTMs used for functional studies mAlbPTM97C2 and mAlbPTM158 were generated by cloning 279 bp and 149 bp BD sequence into the PTM cassette between NheI and SacII sites and were verified by sequencing.

### Mouse Albumin Minigene Target Pre-mRNA

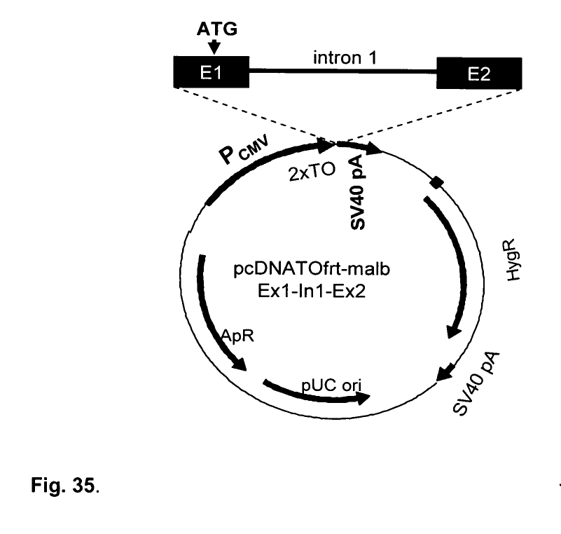

For demonstrating in vitro Apo A-1 function, a mouse albumin mini-gene target consisting of exon 1, intron 1 and exon 2 was used. A schematic diagram of the pre-mRNA target is shown in FIG. 35. The mouse albumin coordinates are as described in Ref Seq. NC_{—}000071. The mouse albumin Ex1-1-n1-Ex2 pre-mRNA target (mAlbEx1-1-n1-Ex2) constructed as follows: 877 bp fragment corresponding to nucleotides 1 through 877 was PCR amplified using the following mouse genomic DNA and primers: mAlb-Ex1F (5′-ctagGCTAGC ACCTTT CCTATCAACCCCACTAGC) and mAlb8 (5′-ctagGCGGCCGC AGGCCTTTGAAATGTTGTTCTCC). These primers contain unique restriction sites at the end of the fragment (indicated in bold). The PCR product was digested with Nhe I and Not I and cloned into inducible expression vector pcDNA5/FRT/TO designed to use with Flip-In T-Rex system (Invitrogen). The final construct (pcDNATOfrt-mAlbEx1-1-n1-Ex2) contains the following features: CMV promoter, Tet operator, SV40 polyadenylation site and hygromycin selection marker for establishing stable cell lines.

### Generation of a Stable Cell Line Expressing Albumin Target

Using the target plasmid described above, a stable target cell line that expressed the mouse albumin mini-gene target consist of exon 1, intron 1 and exon 2 was generated. Analysis of total RNA from cells transfected with target plasmid (pcDNATOfrt-mAlbEx1-1-n1-Ex2) by RT-PCR produced the expected cis-spliced product, but no albumin protein. Upon confirming the splicing pattern of mouse albumin mini-gene target pre-mRNA, a stable cell line in Flip-In T-Rex 293 cells was established by transfecting the target plasmid followed by hygromycin selection. After selecting for a period of ˜2 weeks, hygromycin resistant clones were pooled and maintained in hygromycin until used.

### Efficient Trans-Splicing of Human Apo A1 PTMs

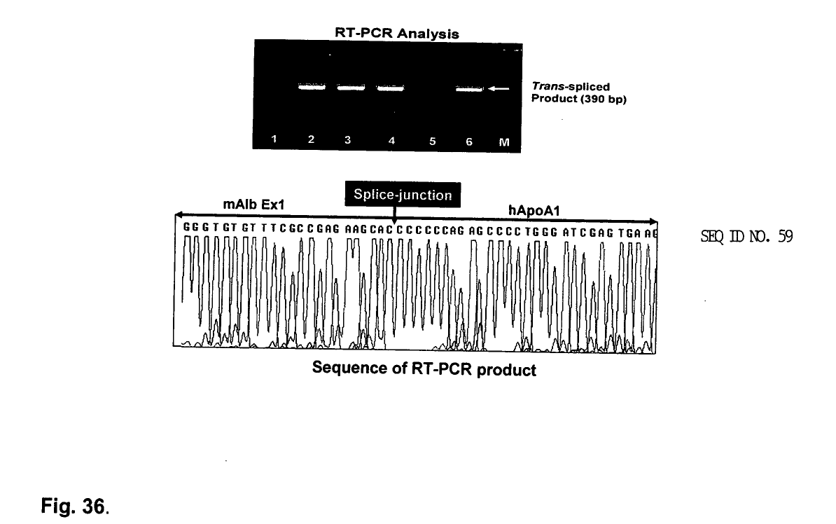

Human Apo A-1 PTMs selected from the HCS shows efficient and accurate trans-splicing to mouse albumin pre-mRNA in stable cells. PTM mediated trans-splicing and production of mouse albumin-human Apo A-1 chimeric mRNA was evaluated by transfecting stable cells with mAlbPTM97C2 and mAlbPTM158, along with a splice mutant lacking the TSD (splice incompetent PTM) and mock transfection. Total RNA isolated from these cells was analyzed by RT-PCR using mouse albumin target and human Apo A-1 PTM specific primers. These primers produced the predicted 390 bp product only in cells that received functional PTMs (FIG. 36, lanes 2-4 and 6). No such product was detected in cells transfected with the splice mutant or in mock transfection (FIG. 36, lane 1 and 5). The PCR product was purified and was directly sequenced, confirming the precise trans-splicing to the predicted splice sites of the PTM and the target pre-mRNA in stable cells (FIG. 36).

Real-time quantitative RT-PCR was used to quantify the fraction of mouse albumin pre-mRNA transcripts converted into chimeric mRNAs by PTMs. Primers for real-time qPCR were designed to discriminate between target exon 1 and trans-spliced mRNAs. Using the protocols described previously, trans-splicing efficiency of mAlbPTM97C2 and mAlbPTM158 was quantified.

Mouse albumin specific PTMs 97C2 and 158 showed a trans-splicing efficiency of 5.6% and 3.45%, respectively. These data confirmed robust trans-splicing between mouse albumin mini-gene target pre-mRNA and PTMs in stable cells.

### Trans-Splicing and Production of Full-Length Protein

The PTM-mediated trans-splicing was assessed for the ability to produce full-length mouse albumin-human Apo A-1 fusion protein in stable cells. Briefly, assay cells expressing the mouse albumin mini-gene pre-mRNA was transfected with mAlbPTMs (97C2 and 158), human albumin-Apo A-1 fusion as a positive control, and splice mutant with a point mutation (G>T) at splice junction as a negative control. Cells were washed after 5 hrs with serum free media and incubated with advanced DMEM serum free media. After 48 hrs, the media was collected, concentrated and analyzed by Western blot. Production of full-length human Apo A-1 protein was demonstrated using anti-human Apo A-1 antibody as described above.

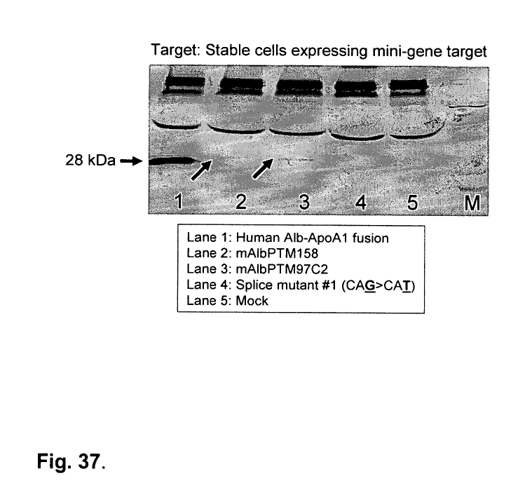

Accurate trans-splicing between mouse albumin exon 1 and PTM would result in a 28 kDa albumin-human Apo A-1 fusion protein. Trans-splicing mediated production of full-length mature human Apo A-1 protein is evident in cells transfected with functional PTMs (97C2 and 158) (FIG. 37, lanes 2-3) but not in controls i.e., cells transfected with a splice mutant or in mock (FIG. 37, lanes 4-5) and it also co-migrated with the albumin-Apo A-1 fusion protein produced using cDNA control plasmid (FIG. 37, lane 1-3). These studies again confirmed precise trans-splicing between the mouse albumin exon 1 and human Apo A-1 PTMs, resulting in the production of fusion albumin-human Apo A-1 protein in stable cells.

### EXAMPLE: TRANS-SPLICING TO ENDOGENOUS MOUSE ALBUMIN PRE-mRNA IN MICE

The efficacy of the lead PTMs selected from the high capacity screen (HCS) were evaluated in vivo. Fifty micrograms of mAlbPTM97C2 (PTM only) or 20 μg of mouse albumin mini-gene target plus 30 μg of mAlbPTM97C2 plasmids were mixed with jet-PEI-Gal (Q-Biogen) reagent and injected via tail vein into normal C57BL/6 mice. Liver and serum samples were collected at 24 and 48 hrs time points. Total and poly A mRNA was isolated and analyzed by RT-PCR using mouse albumin exon 1 specific and human Apo A-1 PTM specific primers.

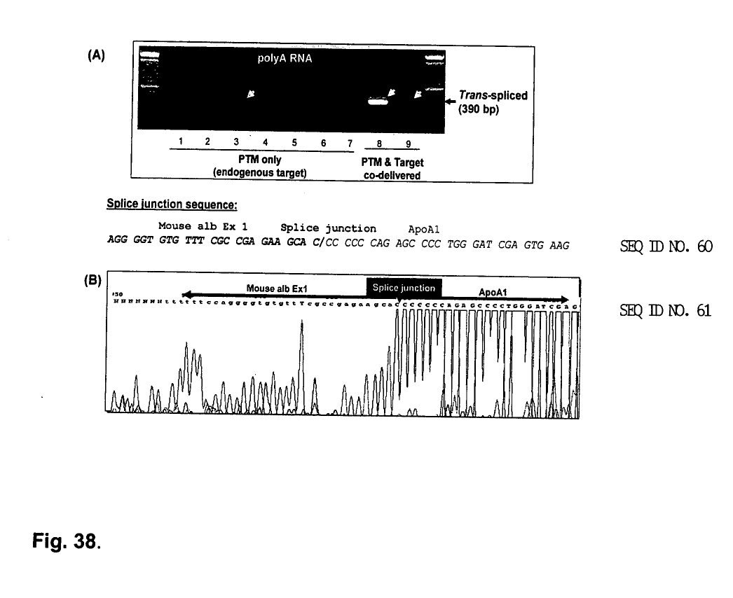

Trans-splicing was detected in a single round in mice that received both mini-gene target plus PTM plasmids, as well as in mice that received PTM only (FIG. 38, lane 3, 8 & 9). Each positive RT-PCR product was purified and sequenced demonstrating the precise trans-splicing of mouse albumin exon 1 into human Apo A-1 coding sequence at the predicted splice sites (FIG. 38, lower panel). These results demonstrated accurate trans-splicing between the PTM and the endogenous albumin pre-mRNA target in mice and further validated albumin targeting strategy in vivo.

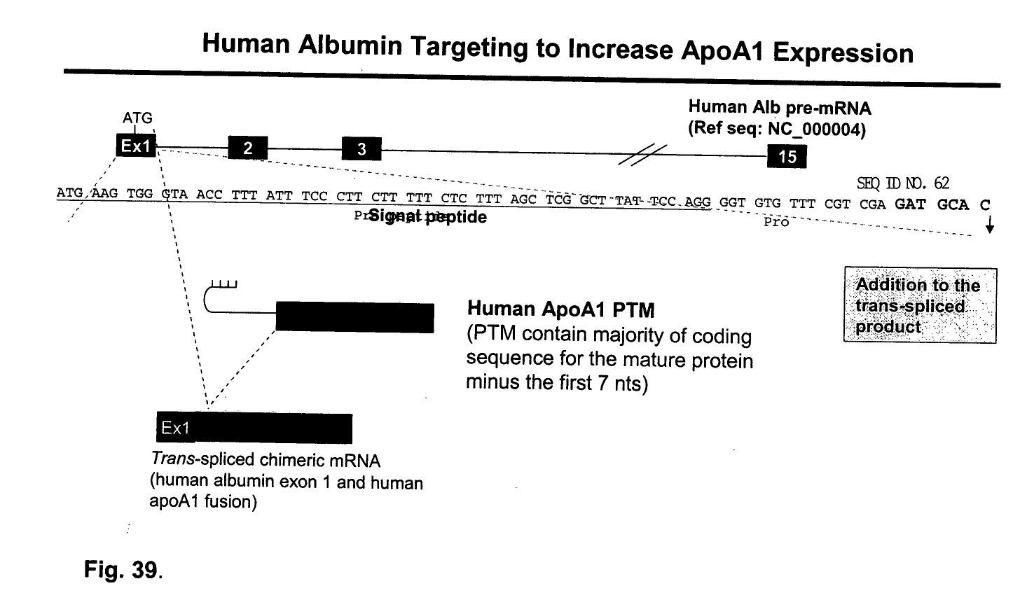

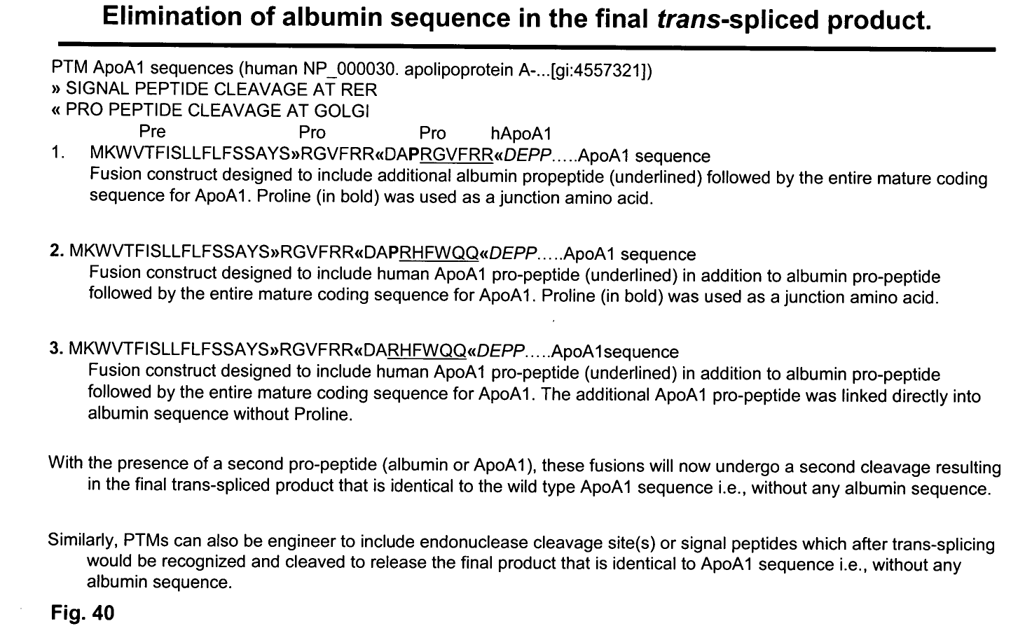

FIG. 39 describes a strategy to increase ApoA1 expression by targeting to human albumin sequences. FIG. 40 describes various means of eliminating albumin sequences in the final trans-spliced product, i.e. to produce a trans-spliced product that is identical to the wild type human ApoA1 without any albumin sequence.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A cell comprising a nucleic acid molecule wherein said nucleic acid molecule comprises:
a) one or more target binding domains that target binding of the nucleic acid molecule to a target pre-mRNAs expressed within the cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

2. A cell comprising a nucleic acid molecule wherein said nucleic acid molecule comprises:
a) one or more target binding domains that target binding of the nucleic acid molecule to a target pre-mRNAs expressed within the cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

3. A cell comprising a nucleic acid molecule wherein said nucleic acid molecule comprises:
a) one or more target binding domains that target binding of the nucleic acid molecule to a target pre-mRNA expressed within the cell;
b) a 5′ splice site;
c) a spacer region that separates the 5′ splice site from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

4. The cell of claim 1 wherein the nucleic acid molecule further comprises a 5′ donor site.

5. The cell of claim 1 wherein the 3′ splice region further comprises a pyrimidine tract.

6. The cell of claim 1, 2 or 3 wherein said nucleic acid molecule further comprises a safety sequence comprising one or more complementary sequences that bind to one or both sides of the 5′ splice site.

7. The cell of claim 1, 2 or 3 wherein the target pre-mRNA expressed within the cell is a human apoA-1 target.

8. The cell of claim 1, 2 or 3 wherein the target pre-mRNA expressed within the cell is a human apoB target.

9. The cell of claim 1, 2 or 3 wherein the target pre-mRNA expressed within the cell is a human albumin target.

10. The cell of claim 1, 2 or 3 wherein the target pre-mRNA is expressed within a liver cell.

11. The cell of claim 1, 2 or 3 wherein the apoA-1 variant is apoA-1 Milano.

12. The cell of claim 1, 2 or 3 wherein the nucleic acid molecule further comprises a safety nucleotide sequence comprising one or more complementary sequences that bind to one or more sides of the 3′ splice region.

13. A cell comprising a recombinant vector wherein said vector expresses a nucleic acid molecule comprising:
a) one or more target binding domains that target binding of the nucleic acid molecule to a target pre-mRNAs expressed within the cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide
wherein said nucleic acid molecule is recognized by nuclear splicing components within the cell.

14. A cell comprising a recombinant vector wherein said vector expresses a nucleic acid molecule comprising:
a) one or more target binding domains that target binding of the nucleic acid molecule to a target pre-mRNAs expressed within the cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

15. A cell comprising a recombinant vector wherein said vector expresses a nucleic acid molecule comprising:
a) one or more target binding domains that target binding of the nucleic acid molecule to a target pre-mRNAs expressed within the cell;
b) a 5′ splice site;
c) a spacer region that separates the 5′ splice site from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

16. The cell of claim 13 wherein the nucleic acid molecule further comprises a 5′ donor site.

17. The cell of claim 13 wherein the 3′ splice region further comprises a pyrimidine tract.

18. The cell of claim 13, 14 or 15 wherein the nucleic acid molecule further comprises a safety nucleotide sequence comprising one or more complementary sequences that bind to one or more sides of the 3′ splice region.

19. The cell of claim 13, 14 or 15 wherein the target pre-mRNA expressed within the cell is a human apoA-1 target.

20. The cell of claim 13, 14 or 15 wherein the target pre-mRNA expressed within the cell is a human apoB target.

21. The cell of claim 13, 14 or 15, wherein the target pre-mRNA expressed within the cell is a human albumin target.

22. The cell of claim 13, 14 or 15 wherein the target pre-mRNA is expressed within a liver cell.

23. The cell of claim 13, 14 or 15 wherein the apoA-1 variant is apoA-1 Milano.

24. A method of producing a chimeric RNA molecule in a cell comprising:
contacting a target target pre-mRNAs expressed in the cell with a nucleic acid molecule recognized by nuclear splicing components wherein said nucleic acid molecule comprises:
a) one or more target binding domains that target binding of the nucleic acid molecule to a target pre-mRNAs expressed within the cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
under conditions in which a portion of the nucleic acid molecule is trans-spliced to a portion of the target pre-mRNA to form a chimeric RNA within the cell.

25. A method of producing a chimeric RNA molecule in a cell comprising:
contacting target pre-mRNAs expressed in the cell with a nucleic acid molecule recognized by nuclear splicing components wherein said nucleic acid molecule comprises:
a) one or more target binding domains that target binding of the nucleic acid molecule to target pre-mRNAs expressed within the cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
under conditions in which a portion of the nucleic acid molecule is trans-spliced to a portion of the target pre-mRNA to form a chimeric RNA within the cell.

26. A method of producing a chimeric RNA molecule in a cell comprising:
contacting a target pre-mRNA expressed within the cell with a nucleic acid molecule recognized by nuclear splicing components wherein said nucleic acid molecule comprises:
a) one or more target binding domains that target binding of the nucleic acid molecule to target pre-mRNAs expressed within the cell;
b) a 5′ splice site;
c) a spacer region that separates the 5′ splice site from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

27. The method of claim 24 wherein the nucleic acid molecule further comprises a 5′ donor site.

28. The method of claim 24 wherein the 3′ splice region further comprises a pyrimidine tract.

29. The method of claim 24, 25 or 26 wherein the nucleic acid molecule further comprises a safety nucleotide sequence comprising one or more complementary sequences that bind to one or more sides of the 3′ splice region.

30. The method of claim 24, 25 or 26 wherein the target pre-mRNA expressed within the cell is a human apoA-1 target.

31. The method of claim 24, 25 or 26 wherein the target pre-mRNA expressed within the cell is a human apoB target.

32. The method of claim 24, 25 or 26 wherein the target pre-mRNA expressed within the cell is a human albumin target.

33. The method of claim 24, 25 or 26 wherein the target pre-mRNA is expressed within a liver cell.

34. The method of claim 24, 25 or 26 wherein the apoA-1 variant is apoA-1 Milano.

35. A nucleic acid molecule comprising:
a) one or more target binding domains that target binding of the nucleic acid molecule to target pre-mRNAs expressed within a cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

36. A nucleic acid molecule comprising:
a) one or more target binding domains that target binding of the nucleic acid molecule to target pre-mRNAs expressed within a cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

37. A nucleic acid molecule comprising:
a) one or more target binding domains that target binding of the nucleic acid molecule to target pre-mRNAs expressed within a cell;
b) a 5′ splice site;
c) a spacer region that separates the 5′ splice site from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

38. The nucleic acid molecule of claim 35 wherein the nucleic acid molecule further comprises a 5′ donor site.

39. The nucleic acid molecule of claim 35 wherein the 3′ splice region further comprises a pyrimidine tract.

40. The nucleic acid molecule of claim 35, 36 or 37 wherein the nucleic acid molecule further comprises a safety nucleotide sequence comprising one or more complementary sequences that bind to one or more sides of the 3′ splice region.

41. The nucleic acid molecule of claim 35, 36 or 37 wherein the target pre-mRNA expressed within the cell is a human apoA-1 target.

42. The nucleic acid molecule of claim 35, 36 or 37 wherein the target pre-mRNA expressed within the cell is a human apoB target.

43. The nucleic acid molecule of claim 35, 36 or 37 wherein the target pre-mRNA expressed within the cell is a human albumin target.

44. The nucleic acid molecule of claim 35, 36 or 37 wherein the target pre-mRNA is expressed within a liver cell.

45. The nucleic acid molecule of claim 35, 36 or 37 wherein the apoA-1 variant is apoA-1 Milano.

46. A eukaryotic expression vector wherein said vector expresses a nucleic acid molecule comprising:
a) one or more target binding domains that target binding of the nucleic acid molecule to target pre-mRNAs expressed within a cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

47. A eukaryotic expression vector wherein said vector expresses a nucleic acid molecule comprising:
a) one or more target binding domains that target binding of the nucleic acid molecule to target pre-mRNAs expressed within a cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

48. A eukaryotic expression vector wherein said vector expresses a nucleic acid molecule comprising:
a) one or more target binding domains that target binding of the nucleic acid molecule to pre-mRNAs expressed within a cell;
b) a 5′ splice site;
c) a spacer region that separates the 5′ splice site from the target binding domain; and
d) a nucleotide sequence to be trans-spliced to the target pre-mRNA wherein said nucleotide sequence encodes an apoA-1 Milano variant polypeptide;
wherein said nucleic acid molecule is recognized by nuclear splicing components within the cell.

49. The vector of claim 46 wherein the nucleic acid molecule further comprises a 5′ donor site.

50. The vector of claim 46 wherein the nucleic acid molecule further comprises a pyrimidine tract.

51. The vector of claim 46, 47, or 48 wherein the nucleic acid molecule further comprises a safety nucleotide sequence comprising one or more complementary sequences that bind to one or more sides of the 3′ splice region.

52. The vector of claim 46, 47, or 48 wherein the target pre-mRNA expressed within the cell is a human apoA-1 target.

53. The vector of claim 46, 47, or 48 wherein the target pre-mRNA expressed within the cell is a human apoB target.

54. The vector of claim 46, 47, or 48 wherein the target pre-mRNA expressed within the cell is a human albumin target.

55. The vector of claim 46, 47, or 48 wherein the target pre-mRNA is expressed within a liver cell.

56. The vector of claim 46, 47, or 48 wherein the apoA-1 variant is apoA-1 Milano.

57. The vector of claim 46, 47, or 48 wherein said vector is a viral vector.

58. The vector of claim 46, 47, or 48 wherein expression of the nucleic acid molecule is controlled by a liver cell specific promoter.

59. A method for expressing an apoA-1 variant in a subject comprising administering to said subject a nucleic acid molecule comprising:
a) one or more target binding domains that target binding of the nucleic acid molecule to target pre-mRNAs expressed within a cell; and
b) a nucleotide sequence to be trans-spliced to the target pre-mRNA wherein said nucleotide sequence encodes an apoA-1 variant polypeptide;
wherein said nucleic acid molecule is recognized by nuclear splicing components within the cell.

60. The method of claim 59 wherein the target pre-mRNA expressed within the cell is a human apoA-1 target.

61. The method of claim 59 wherein the target pre-mRNA expressed within the cell is a human apoB target.

62. The method of claim 59 wherein the target pre-mRNA expressed within the cell is a human albumin target.

63. The method of claim 54 wherein the target pre-mRNA is expressed within a liver cell.

64. The method of claim 54 wherein the apoA-1 variant is apoA-1 Milano.

65. A cell comprising a nucleic acid molecule wherein said nucleic acid molecule comprises:
a) one or more target binding domains that target binding of the nucleic acid molecule to a target mRNAs expressed within the cell;
b) a sequence having ribozyme activity; and
c) a nucleotide sequence to be trans-spliced to the target mRNA wherein said nucleotide sequence encodes an apoA-1 variant polypeptide.

66. The cell of claim 65 wherein the target mRNA expressed within the cell is a human apoA-1 target.

67. The cell of claim 65 wherein the target mRNA expressed within the cell is a human apoB target.

68. The cell of claim 65 wherein the target pre-mRNA expressed within the cell is a human albumin target.

69. The cell of claim 65 wherein the target mRNA is expressed within a liver cell.

70. The cell of claim 65 wherein the apoA-1 variant is apoA-1 Milano.

71. A cell comprising a recombinant vector wherein said vector expresses a nucleic acid molecule comprising:
a) one or more target binding domains that target binding of the nucleic acid molecule to a target mRNAs expressed within the cell;
b) a sequence having ribozyme activity; and
c) a nucleotide sequence to be trans-spliced to the target mRNA wherein said nucleotide sequence encodes an apoA-1 variant polypeptide.

72. The cell of claim 71 wherein the target mRNA expressed within the cell is a human apoA-1 target.

73. The cell of claim 71 wherein the target mRNA expressed within the cell is a human apoB target.

74. The cell of claim 71 wherein the target pre-mRNA expressed within the cell is a human albumin target.

75. The cell of claim 71 wherein the target mRNA is expressed within a liver cell.

76. The cell of claim 71 wherein the apoA-1 variant is apoA-1 Milano.

77. A method of producing a chimeric RNA molecule in a cell comprising:
contacting a target target mRNAs expressed in the cell with a nucleic acid molecule wherein said nucleic acid molecule comprises:
a) one or more target binding domains that target binding of the nucleic acid molecule to a target mRNAs expressed within the cell;
b) a sequence having ribozyme activity; and
c) a nucleotide sequence to be trans-spliced to the target mRNA wherein said nucleotide sequence encodes an apoA-1 variant polypeptide;
under conditions in which a portion of the nucleic acid molecule is trans-spliced to a portion of the target mRNA to form a chimeric RNA within the cell.

78. The method of claim 77 wherein the target mRNA expressed within the cell is a human apoA-1 target.

79. The method of claim 77 wherein the target mRNA expressed within the cell is a human apoB target.

80. The cell of claim 77 wherein the target pre-mRNA expressed within the cell is a human albumin target.

81. The method of claim 77 wherein the target mRNA is expressed within a liver cell.

82. The method of claim 77 wherein the apoA-1 variant is apoA-1 Milano.

83. A nucleic acid molecule comprising:
a) one or more target binding domains that target binding of the nucleic acid molecule to target mRNAs expressed within a cell;
b) a sequence having ribozyme activity; and
c) a nucleotide sequence to be trans-spliced to the target mRNA wherein said nucleotide sequence encodes an apoA-1 variant polypeptide.

84. The nucleic acid molecule of claim 83 wherein the target mRNA expressed within the cell is a human apoA-1 target.

85. The nucleic acid molecule of claim 83 wherein the target mRNA expressed within the cell is a human apoB target.

86. The nucleic acid molecule of claim 83 wherein the target pre-mRNA expressed within the cell is a human albumin target.

87. The nucleic acid molecule of claim 83 wherein the target mRNA is expressed within a liver cell.

88. The nucleic acid molecule of claim 83 wherein the apoA-1 variant is apoA-1 Milano.

89. A eukaryotic expression vector wherein said vector expresses a nucleic acid molecule comprising:
a) one or more target binding domains that target binding of the nucleic acid molecule to target mRNAs expressed within a cell;
b) a sequence having ribozyme activity; and
c) a nucleotide sequence to be trans-spliced to the target mRNA wherein said nucleotide sequence encodes an apoA-1 variant polypeptide.

90. The vector of claim 89 wherein the target mRNA expressed within the cell is a human apoA-1 target.

91. The vector of claim 89 wherein the target mRNA expressed within the cell is a human apoB target.

92. The vector of claim 89 wherein the target pre-mRNA expressed within the cell is a human albumin target.

93. The vector of claim 89 wherein the target mRNA is expressed within a liver cell.

94. The vector of claim 89 wherein the apoA-1 variant is apoA-1 Milano.

95. The vector of claim 89 wherein said vector is a viral vector.

96. The vector of claim 89 wherein expression of the nucleic acid molecule is controlled by a liver cell specific promoter.

97. A method for expressing an apoA-1 variant in a subject comprising administering to said subject a nucleic acid molecule comprising:
a) one or more target binding domains that target binding of the nucleic acid molecule to target mRNAs expressed within a cell;
b) a sequence having ribozyme activity; and
c) a nucleotide sequence to be trans-spliced to the target mRNA wherein said nucleotide sequence encodes an apoA-1 variant polypeptide.

98. The method of claim 97 wherein the target mRNA expressed within the cell is a human apoA-1 target.

99. The method of claim 97 wherein the target mRNA expressed within the cell is a human apoB target.

100. The method of claim 97 wherein the target pre-mRNA expressed within the cell is a human albumin target.

101. The method of claim 97 wherein the target mRNA is expressed within a liver cell.

102. The method of claim 97 wherein the apoA-1 variant is apoA-1 Milano.

103. A cell comprising a nucleic acid molecule wherein said nucleic acid molecule comprises:
a) one or more target binding domains that target binding of the nucleic acid molecule to an albumin target pre-mRNAs expressed within the cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type or apoA-1 variant polypeptide;
wherein said nucleic acid molecule is recognized by nuclear splicing components within the cell.

104. A cell comprising a nucleic acid molecule wherein said nucleic acid molecule comprises:
a) one or more target binding domains that target binding of the nucleic acid molecule to an albumin target pre-mRNAs expressed within the cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes encodes a wild type apo-A1 or apoA-1 variant polypeptide;
wherein said nucleic acid molecule is recognized by nuclear splicing components within the cell.

105. A cell comprising a nucleic acid molecule wherein said nucleic acid molecule comprises:
a) one or more target binding domains that target binding of the nucleic acid molecule to an albumin target pre-mRNA expressed within the cell;
b) a 5′ splice site;
c) a spacer region that separates the 5′ splice site from the target binding domain; and
d) a nucleotide sequence to be trans-spliced to the target pre-mRNA wherein said nucleotide sequence encodes a wild type apo-A1 or apoA-1 variant polypeptide;
wherein said nucleic acid molecule is recognized by nuclear splicing components within the cell.

106. The cell of claim 103 wherein the nucleic acid molecule further comprises a 5′ donor site.

107. The cell of claim 103 wherein the 3′ splice region further comprises a pyrimidine tract.

108. The cell of claim 103, 104 or 105 wherein said nucleic acid molecule further comprises a safety sequence comprising one or more complementary sequences that bind to one or both sides of the 5′ splice site.

109. The cell of claim 103, 104 or 105 wherein the apoA-1 variant is apoA-1 Milano.

110. The cell of claim 103, 104 or 105 wherein the nucleic acid molecule further comprises a safety nucleotide sequence comprising one or more complementary sequences that bind to one or more sides of the 3′ splice region.

111. A cell comprising a recombinant vector wherein said vector expresses a nucleic acid molecule comprising:
a) one or more target binding domains that target binding of the nucleic acid molecule to albumin target pre-mRNAs expressed within the cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apo-A1 or apoA-1 variant polypeptide
wherein said nucleic acid molecule is recognized by nuclear splicing components within the cell.

112. A cell comprising a recombinant vector wherein said vector expresses a nucleic acid molecule comprising:
a) one or more target binding domains that target binding of the nucleic acid molecule to albumin target pre-mRNAs expressed within the cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apoA1 or apoA-1 variant polypeptide;
wherein said nucleic acid molecule is recognized by nuclear splicing components within the cell.

113. A cell comprising a recombinant vector wherein said vector expresses a nucleic acid molecule comprising:
a) one or more target binding domains that target binding of the nucleic acid molecule to albumin target pre-mRNAs expressed within the cell;
b) a 5′ splice site;
c) a spacer region that separates the 5′ splice site from the target binding domain; and
d) a nucleotide sequence to be trans-spliced to the target pre-mRNA wherein said nucleotide sequence encodes a wild type apoA1 or apoA-1 variant polypeptide;
wherein said nucleic acid molecule is recognized by nuclear splicing components within the cell.

114. The cell of claim 111 wherein the nucleic acid molecule further comprises a 5′ donor site.

115. The cell of claim 111 wherein the 3′ splice region further comprises a pyrimidine tract.

116. The cell of claim 111, 112, or 113 wherein the nucleic acid molecule further comprises a safety nucleotide sequence comprising one or more complementary sequences that bind to one or more sides of the 3′ splice region.

117. The cell of claim 111, 112, or 113 wherein the apoA-1 variant is apoA-1 Milano.

118. A method of producing a chimeric RNA molecule in a cell comprising:
contacting albumin target pre-mRNAs expressed in the cell with a nucleic acid molecule recognized by nuclear splicing components wherein said nucleic acid molecule comprises:
a) one or more target binding domains that target binding of the nucleic acid molecule to albumin target pre-mRNAs expressed within the cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apo-A1 or apoA-1 variant polypeptide;
under conditions in which a portion of the nucleic acid molecule is trans-spliced to a portion of the target pre-mRNA to form a chimeric RNA within the cell.

119. A method of producing a chimeric RNA molecule in a cell comprising:
contacting target pre-mRNAs expressed in the cell with a nucleic acid molecule recognized by nuclear splicing components wherein said nucleic acid molecule comprises:
a) one or more target binding domains that target binding of the nucleic acid molecule to albumin target pre-mRNAs expressed within the cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apo-A1 or apoA-1 variant polypeptide;
under conditions in which a portion of the nucleic acid molecule is trans-spliced to a portion of the target pre-mRNA to form a chimeric RNA within the cell.

120. A method of producing a chimeric RNA molecule in a cell comprising:
contacting an albumin target pre-mRNA expressed within the cell with a nucleic acid molecule recognized by nuclear splicing components wherein said nucleic acid molecule comprises:
a) one or more target binding domains that target binding of the nucleic acid molecule to albumin target pre-mRNAs expressed within the cell;
b) a 5′ splice site;
c) a spacer region that separates the 5′ splice site from the target binding domain; and
d) a nucleotide sequence to be trans-spliced to the target pre-mRNA wherein said nucleotide sequence encodes a wild type apo-A1 or apoA-1 variant polypeptide;
wherein said nucleic acid molecule is recognized by nuclear splicing components within the cell.

121. The method of claim 118 wherein the nucleic acid molecule further comprises a 5′ donor site.

122. The method of claim 118 wherein the 3′ splice region further comprises a pyrimidine tract.

123. The method of claim 118, 119 or 120 wherein the nucleic acid molecule further comprises a safety nucleotide sequence comprising one or more complementary sequences that bind to one or more sides of the 3′ splice region.

124. The method of claim 118, 119 or 120 wherein the apoA-1 variant is apoA-1 Milano.

125. A nucleic acid molecule comprising:
a) one or more target binding domains that target binding of the nucleic acid molecule to albumin target pre-mRNAs expressed within a cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apo-A1 or apoA-1 variant polypeptide;
wherein said nucleic acid molecule is recognized by nuclear splicing components within the cell.

126. A nucleic acid molecule comprising:
a) one or more target binding domains that target binding of the nucleic acid molecule to albumin target pre-mRNAs expressed within a cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes an a wild type apoA-1 or apoA-1 variant polypeptide;
wherein said nucleic acid molecule is recognized by nuclear splicing components within the cell.

127. A nucleic acid molecule comprising:
a) one or more target binding domains that target binding of the nucleic acid molecule to albumin target pre-mRNAs expressed within a cell;
b) a 5′ splice site;
c) a spacer region that separates the 5′ splice site from the target binding domain; and
d) a nucleotide sequence to be trans-spliced to the target pre-mRNA wherein said nucleotide sequence encodes a wild type apo-A1 or apoA-1 variant polypeptide; wherein said nucleic acid molecule is recognized by nuclear splicing components within the cell.

128. The nucleic acid molecule of claim 125 wherein the nucleic acid molecule further comprises a 5′ donor site.

129. The nucleic acid molecule of claim 125 wherein the 3′ splice region further comprises a pyrimidine tract.

130. The nucleic acid molecule of claim 125, 126 or 127 wherein the nucleic acid molecule further comprises a safety nucleotide sequence comprising one or more complementary sequences that bind to one or more sides of the 3′ splice region.

131. The nucleic acid molecule of claim 125, 126 or 127 wherein the apoA-1 variant is apoA-1 Milano.

132. A eukaryotic expression vector wherein said vector expresses a nucleic acid molecule comprising:
a) one or more target binding domains that target binding of the nucleic acid molecule to albumin target pre-mRNAs expressed within a cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apo-A1 or apoA-1 variant polypeptide;
wherein said nucleic acid molecule is recognized by nuclear splicing components within the cell.

133. A eukaryotic expression vector wherein said vector expresses a nucleic acid molecule comprising:
a) one or more target binding domains that target binding of the nucleic acid molecule to albumin target pre-mRNAs expressed within a cell;
b) a 3′ splice acceptor site;
c) a spacer region that separates the 3′ splice region from the target binding domain; and
d) a nucleotide sequence to be trans-spliced to the target pre-mRNA wherein said nucleotide sequence encodes a wild type apo-A1 or apoA-1 variant polypeptide;
wherein said nucleic acid molecule is recognized by nuclear splicing components within the cell.

134. A eukaryotic expression vector wherein said vector expresses a nucleic acid molecule comprising:
a) one or more target binding domains that target binding of the nucleic acid molecule to an albumin pre-mRNA expressed within a cell;
b) a 5′ splice site;
c) a spacer region that separates the 5′ splice site from the target binding domain; and
d) a nucleotide sequence to be trans-spliced to the target pre-mRNA wherein said nucleotide sequence encodes a wild type apo-A1 or apoA-1 variant polypeptide;
wherein said nucleic acid molecule is recognized by nuclear splicing components within the cell.

135. The vector of claim 132 wherein the nucleic acid molecule further comprises a 5′ donor site.

136. The vector of claim 132 wherein the nucleic acid molecule further comprises a pyrimidine tract.

137. The vector of claim 132, 133, or 134 wherein the nucleic acid molecule further comprises a safety nucleotide sequence comprising one or more complementary sequences that bind to one or more sides of the 3′ splice region.

138. The vector of claim 132, 133, or 134 wherein the apoA-1 variant is apoA-1 Milano.

139. The vector of claim 132, 133, or 134 wherein said vector is a viral vector.

140. The vector of claim 132, 133, or 134 wherein expression of the nucleic acid molecule is controlled by a liver cell specific promoter.

141. A method for expressing an apoA-1 variant in a subject comprising administering to said subject a nucleic acid molecule comprising:
a) one or more target binding domains that target binding of the nucleic acid molecule to an albumin target pre-mRNA expressed within a cell; and
b) a nucleotide sequence to be trans-spliced to the target pre-mRNA wherein said nucleotide sequence encodes a wild type apo-A1 or apoA-1 variant polypeptide; wherein said nucleic acid molecule is recognized by nuclear splicing components within the cell.

142. The method of claim 141 wherein the apoA-1 variant is apoA-1 Milano.

143. A cell comprising a nucleic acid molecule wherein said nucleic acid molecule comprises:
a) one or more target binding domains that target binding of the nucleic acid molecule to albumin target mRNAs expressed within the cell;
b) a sequence having ribozyme activity; and
c) a nucleotide sequence to be trans-spliced to the target mRNA wherein said nucleotide sequence encodes a wild type apo-A1 or apoA-1 variant polypeptide.

144. The cell of claim 143 wherein the apoA-1 variant is apoA-1 Milano.

145. A cell comprising a recombinant vector wherein said vector expresses a nucleic acid molecule comprising:
a) one or more target binding domains that target binding of the nucleic acid molecule to albumin target mRNAs expressed within the cell;
b) a sequence having ribozyme activity; and
c) a nucleotide sequence to be trans-spliced to the target mRNA wherein said nucleotide sequence encodes a wild type apo-A1 or apoA-1 variant polypeptide.

146. The cell of claim 145 wherein the apoA-1 variant is apoA-1 Milano.

147. A method of producing a chimeric RNA molecule in a cell comprising:
contacting a target mRNAs expressed in the cell with a nucleic acid molecule wherein said nucleic acid molecule comprises:
a) one or more target binding domains that target binding of the nucleic acid molecule to albumin target mRNAs expressed within the cell;
b) a sequence having ribozyme activity; and
c) a nucleotide sequence to be trans-spliced to the target mRNA wherein said nucleotide sequence encodes a wild type apo-A1 or apoA-1 variant polypeptide;
under conditions in which a portion of the nucleic acid molecule is trans-spliced to a portion of the target mRNA to form a chimeric RNA within the cell.

148. The method of claim 147 wherein the apoA-1 variant is apoA-1 Milano.

149. A nucleic acid molecule comprising:
a) one or more target binding domains that target binding of the nucleic acid molecule to albumin target mRNAs expressed within a cell;
b) a sequence having ribozyme activity; and
c) a nucleotide sequence to be trans-spliced to the target mRNA wherein said nucleotide sequence encodes a wild type apo-A1 or apoA-1 variant polypeptide.

150. The nucleic acid molecule of claim 149 wherein the apoA-1 variant is apoA-1 Milano.

151. A eukaryotic expression vector wherein said vector expresses a nucleic acid molecule comprising:
a) one or more target binding domains that target binding of the nucleic acid molecule to albumin target mRNAs expressed within a cell;
b) a sequence having ribozyme activity; and
c) a nucleotide sequence to be trans-spliced to the target mRNA wherein said nucleotide sequence encodes a wild type apo-A1 or apoA-1 variant polypeptide.

152. The vector of claim 151 wherein the apoA-1 variant is apoA-1 Milano.

153. The vector of claim 151 wherein said vector is a viral vector.

154. The vector of claim 151 wherein expression of the nucleic acid molecule is controlled by a liver cell specific promoter.

155. A method for expressing an apoA-1 wild type or a variant in a subject comprising administering to said subject a nucleic acid molecule comprising:
a) one or more target binding domains that target binding of the nucleic acid molecule to albumin target mRNAs expressed within a cell;
b) a sequence having ribozyme activity; and
c) a nucleotide sequence to be trans-spliced to the target mRNA wherein said nucleotide sequence encodes a wild type apo-A1 or apoA-1 variant polypeptide.

156. The method of claim 155 wherein the apoA-1 variant is apoA-1 Milano.
